(19) United States
(12) Patent Application Publication
(10) Pub. No.: US 2008/0153818 A1
(43) Pub. Date: Jun. 26, 2008

(54) METHODS FOR PREVENTING INFLAMMATION DURING SURGERY

(76) Inventor: David P. Bingaman, Weatherford, TX (US)

Correspondence Address: Teresa J. Schultz;IP Legal (TB4-8), Alcon Research, Ltd., 6201 South Freeway, Fort Worth, TX, 76134-2099, US

(21) Appl. No.: 11/962,739

(22) Filed: Dec. 21, 2007

Related U.S. Application Data
(60) Provisional application No. 60/871,407, filed on Dec. 21, 2006.

(51) Int. Cl. A61K 31/5377; A61K 31/423; A61P 27/02; A61P 29/00

(52) U.S. Cl. 514/233.8; 514/379

(57) ABSTRACT

The present invention provides compositions and methods for preventing inflammation and retinal edema resulting from an ocular surgical procedure by administering to a patient undergoing surgery a therapeutically effective amount of a composition comprising an active agent selected from the group consisting of RTK inhibitors and anti-VEGF compounds, and a pharmaceutically acceptable carrier, wherein the RTK inhibitor blocks tyrosine autophosphorylation of VEGFR-1, VEGFR-2, VEGFR-3, Tie-2, PDGFR, c-KIT, Flt-3, and CSF-1R.

### RELATED APPLICATION

The present application claims priority to U.S. Provisional Patent Application No. 60/871,407 filed Dec. 21, 2006.

### BACKGROUND OF THE INVENTION

1. Field of the Invention

The present invention relates to the field of ophthalmology. More particularly, the invention relates to a method for preventing inflammation and retinal edema during surgery, while maintaining the integrity, stability, and function of ocular tissues.

2. Description of the Related Art

The number of surgical techniques and associated products has grown dramatically in all fields of surgery over the past decade. For example, cataract surgery, which is a very delicate operation involving replacement of the natural crystallin lens of the human eye with an artificial lens, was previously considered to be a major surgical procedure requiring hospitalization of the patient and a significant recovery period, but today this procedure is routinely performed on an out-patient basis and enables vision to be restored almost immediately. Similar advancements have been achieved in other areas of ophthalmic surgery—and in other types of surgical procedures.

These remarkable advancements are attributable to various factors, including improved equipment for performing the surgeries, improved surgical techniques developed by innovative surgeons, and improved pharmaceutical products which facilitate successful surgery by minimizing the risks of damaging sensitive, irreplaceable tissue during surgery. Many surgical techniques involve the use of cutting instruments to make an incision in the patient's tissue. At the incision cite, the cutting edge of these instruments unavoidably damages several layers of cells on either side of the point of entry. This impairs the ability of the surgical wound to heal without resulting scar tissue. Additionally, inflammation generally accompanies such surgical wounds and can involve clinically significant tissue edema.

Any incision into the human body is detrimental to the human body and invariably produces cellular trauma and subsequent cell loss. The need to keep cell loss to a minimum is particularly crucial during any surgical procedure performed on delicate and irreplaceable tissues, such as the tissues of the eye, nerves, etc. Another significant factor causing cell loss during tissue scission is the traumatic change in environment experienced by the internal cells. Exposure to the atmosphere presents a far different environment for the cells than is provided by the natural fluids in which they are bathed. To stimulate the natural cellular environment and thereby prevent cell damage, exposed tissue during surgery is frequently irrigated in solutions which attempt to approximate natural body fluids. For example, the value of bathing eye tissue during surgery to prevent cell damage has long been recognized. For internal anterior segment tissues, such as the corneal endothelium, the aqueous humor is the natural bathing fluid and, hence, an ophthalmic irrigating solution intended to protect the endothelium should as closely as possible resemble the aqueous humor. Similarly, the vitreous humor is the fluid that bathes the retina and optic nerve head in the posterior segment and any irrigating solution should resemble it as closely as possible.

What is needed is a safe and effective method for preventing the occurrence of inflammation and retinal edema during surgery and to limit the severity of its occurrence after surgery.

### SUMMARY OF THE INVENTION

The present invention overcomes these and other drawbacks of the prior art by providing a method for preventing inflammation and retinal edema resulting from an ocular surgical procedure by administering to a patient undergoing surgery a therapeutically effective amount of a composition comprising an active agent selected from the group consisting of RTK inhibitors and anti-VEGF compounds, and a pharmaceutically acceptable carrier, wherein the RTK inhibitor blocks tyrosine autophosphorylation of VEGFR-1, VEGFR-2, VEGFR-3, Tie-2, PDGFR, c-KIT, Flt-3, and CSF-1R.

### BRIEF DESCRIPTION OF THE DRAWINGS

The following drawing forms part of the present specification and is included to further demonstrate certain aspects of the present invention. The invention may be better understood by reference to this drawing in combination with the detailed description of specific embodiments presented herein.

FIG. 1 Compound 86 inhibits preretinal neoavascularization (NV) following a single intravitreal injection in the rat model of oxygen-induced retinopathy (OIR).

FIG. 2 Compound 86 prevents preretinal neoavascularization (NV) following oral gavage in the rat model of oxygen-induced retinopathy (OIR).

FIG. 3 Compound 86 inhibits laser-induced choroidal neovascularization (CNV) following a single intravitreal injection in the mouse.

FIG. 4 Compound 86 induces regression of existing laser-induced choroidal neovascularization (CNV) following a single intravitreal injection in the mouse.

FIG. 5 Comparison of CNV lesions between Compound 86-treated groups in the mouse.

FIG. 6 Compound 86 inhibits laser-induced choroidal neovascularization (CNV) following oral gavage in the mouse.

FIG. 7 Compound 86 inhibits diabetes-induced retinal vascular permeability following a single intravitreal injection in the rat.

FIG. 8 Compound 86 inhibits VEGF-induced retinal vascular permeability following a single intravitreal injection in the rat.

FIG. 9 Compound 86 completely prevents diabetes-induced retinal vascular permeability following oral gavage in the STZ rat model.

### DETAILED DESCRIPTION PREFERRED EMBODIMENTS

Inflammation typically occurs when a living body sustains some kind of injury. The injury may be caused by a number of agents, such as physical agents, chemical substances or biological agents. Excessive heat or cold, pressure, ultraviolet or ionizing irradiation, cuts or abrasions are examples of physical agents that may cause inflammation in a living body. A variety of chemical substances, inorganic or organic, are capable of causing inflammation to a living body. Biological agents that may cause inflammation include viruses, bacteria and other parasites. Surgery on a living body may cause inflammation by any one of these agents, although the physical trauma is the most common inflammation-causing aspect of surgery. Tissue trauma results in sterile inflammation through the release of local cytokines and growth factors, such as PGE2. These inflammatory mediators can be directly vasoactive or they can stimulate the production of other hyperpermeability molecules, such as vascular endothelial growth factor (VEGF). VEGF is an extremely potent vascular permeability factor as well as proangiogenic growth factor. Preclinical models of trauma and wound healing have shown that VEGF can be upregulated in association with increased PGE2 and that the increased VEGF can produce enhanced vascular permeability leading to interstitial edema. VEGF mediates these vasoactive functions through 2 receptor tyrosine kinases (RTK), VEGFR-1 and -2. Small molecule inhibitors of VEGFRs have been shown to inhibit vascular permeability and edema in ocular and nonocular animal models.

It has been discovered that receptor tyrosine kinase (RTK) inhibitors that inhibit certain groups of tyrosine kinase receptors with IC₅₀ values of 250 or less can prevent or cause regression of neovascularization in in vivo models of macular degeneration and/or macular edema. It is believed that such compounds can provide protection from inflammation when administered prior to, during, or after ocular surgical procedures.

It is important that the RTK inhibiting compounds for use in the methods of the invention exhibit a receptor binding profile where multiple receptors in the RTK family are blocked by a single compound. One preferred group of receptors for which tyrosine autophosphorylation is blocked includes VEGF receptor 1 (Flt-1), VEGF receptor 2 (KDR), VEGF receptor 3 (Flt-4), Tie-2, PDGFR, c-KIT, Flt-3, and CSF-1R. Additional preferred binding profiles include the following: a) Tie-2, PDGFR, and VEGF receptor 2 (KDR); b) VEGF receptor 2 (KDR), VEGF receptor 1 (Flt-1), PDGFR, and Tie-2; c) VEGF receptor 2 (KDR), VEGF receptor 1 (Flt-1), and Tie-2; d) VEGF receptor 2 (KDR), VEGF receptor 1 (Flt-1), and PDGFR; e) VEGF receptor 2 (KDR) and Tie-2; f) VEGF receptor 2 (KDR) and PDGFR; and g) VEGF receptor 2 (KDR), Tie-2, and PDGFR.

Preferred RTK inhibitor compounds for use in the methods of the present invention are potent, competitive inhibitors of the ATP binding site for a select group of RTKs. That is, preferred agents simultaneously block tyrosine autophosphorylation of VEGFR-1 (Flt-1), VEGFR-2 (KDR), VEGFR-3 (Flt-4), TIE-2, PDGFR, c-KIT, FLT-3, and CSF-1R, or some combination of two or more of these receptors, at low nM concentrations. Preferably, RTK inhibitor compounds for use in the methods of the invention exhibit an IC₅₀ range between 0.1 nM and 250 nM for each of these receptors. More preferred RTK inhibitor compounds exhibit an IC₅₀ range between 0.1 nM and 100 nM for at least six of these receptors. Most preferred RTK inhibitor compounds possess an IC₅₀ range between 0.1 nM and 10 nM for at least four of these receptors.

In one preferred aspect, for each grouping of receptors listed in a)-g) above, the IC₅₀ value of each receptor in each group will be from 0.1 nM to 200 nM. In another preferred aspect, the IC₅₀ value of each receptor in each group will be from 0.1 nM to 100 nM. In yet another preferred embodiment, at least one receptor in each preferred group of receptors listed in a)-g) above will exhibit an IC₅₀ value of less than 10 nM. In yet another preferred embodiment, two or more receptors in each preferred group of receptors listed in a)-g) above will exhibit an IC₅₀ value of less than 10 nM.

Preferred RTKi compounds for use in the compositions and methods of the invention include, but are not limited to, the compounds listed in Table 1:

TABLE 1

| No. | Compound Name |
|---|---|
| 1 | N-[4-(3-amino-1,2-benzisoxazol-4-yl)phenyl]-N′-(3-methylphenyl)urea |
| 2 | N-[4-(3-amino-1,2-benzisoxazol-4-yl)phenyl]-N′-[2-(trifluoromethyl)phenyl]urea |
| 3 | N-[4-(3-amino-1,2-benzisoxazol-4-yl)phenyl]-N′-(2-fluoro-5-methylphenyl)urea |
| 4 | N-[4-(3-amino-1,2-benzisoxazol-4-yl)phenyl]-N′-[3-(trifluoromethyl)phenyl]urea |
| 5 | N-[4-(3-amino-1,2-benzisoxazol-4-yl)phenyl]-N′-[2-fluoro-5- |
| | (trifluoromethyl)phenyl]urea |
| 6 | N-[4-(3-amino-7-methoxy-1,2-benzisoxazol-4-yl)phenyl]-N′-[2-fluoro-5- |
| | (trifluoromethyl)phenyl]urea |
| 7 | N-[4-(3-amino-7-methoxy-1,2-benzisoxazol-4-yl)phenyl]-N′-(3-methylphenyl)urea |
| 8 | N-[4-(3-amino-7-methoxy-1,2-benzisoxazol-4-yl)phenyl]-N′-[3- |
| | (trifluoromethyl)phenyl]urea |
| 9 | N-[4-(3-amino-7-methoxy-1,2-benzisoxazol-4-yl)phenyl]-N′-(3-chlorophenyl)urea |
| 10 | N-[4-(3-amino-7-methoxy-1,2-benzisoxazol-4-yl)phenyl]-N′-(2-fluoro-5- |
| | methylphenyl)urea |
| 11 | N-{4-[3-amino-7-(4-morpholinylmethyl)-1,2-benzisoxazol-4-yl]phenyl}-N′-[2-fluoro- |
| | 5-(trifluoromethyl)phenyl]urea |
| 12 | N-{4-[3-amino-7-(4-morpholinylmethyl)-1,2-benzisoxazol-4-yl]phenyl}-N′-[3- |
| | (trifluoromethyl)phenyl]urea |
| 13 | N-{4-[3-amino-7-(4-morpholinylmethyl)-1,2-benzisoxazol-4-yl]phenyl}-N′-(3- |
| | chlorophenyl)urea |
| 14 | N-{4-[3-amino-7-(4-morpholinylmethyl)-1,2-benzisoxazol-4-yl]phenyl}-N′-(3- |
| | methylphenyl)urea |
| 15 | N-{4-[3-amino-7-(4-morpholinylmethyl)-1,2-benzisoxazol-4-yl]phenyl}-N′-(2-fluoro- |
| | 5-methylphenyl)urea |
| 16 | N-{4-[3-amino-7-(4-morpholinylmethyl)-1,2-benzisoxazol-4-yl]phenyl}-N′-(3,5- |
| | dimethylphenyl)urea |
| 17 | N-{4-[3-amino-7-(4-morpholinylmethyl)-1,2-benzisoxazol-4-yl]phenyl}-N′-(3- |
| | phenoxyphenyl)urea |
| 18 | N-{4-[3-amino-7-(4-morpholinylmethyl)-1,2-benzisoxazol-4-yl]phenyl}-N′-(3- |
| | bromophenyl)urea |
| 19 | N-(4-{3-amino-7-[2-(4-morpholinyl)ethoxy]-1,2-benzisoxazol-4-yl}phenyl)-N′-[3- |
| | (trifluoromethyl)phenyl]urea |
| 20 | N-(4-{3-amino-7-[2-(4-morpholinyl)ethoxy]-1,2-benzisoxazol-4-yl}phenyl)-N′-(2- |
| | fluoro-5-methylphenyl)urea |
| 21 | N-(4-{3-amino-7-[2-(4-morpholinyl)ethoxy]-1,2-benzisoxazol-4-yl}phenyl)-N′-[2- |
| | fluoro-5-(trifluoromethyl)phenyl]urea |
| 22 | N-(4-{3-amino-7-[2-(4-morpholinyl)ethoxy]-1,2-benzisoxazol-4-yl}phenyl)-N′-(3- |
| | methylphenyl)urea |
| 23 | N-[4-(3-amino-1,2-benzisoxazol-4-yl)phenyl]-N′-(3,5-dimethylphenyl)urea |
| 24 | N-[4-(3-amino-1,2-benzisoxazol-4-yl)phenyl]-N′-phenylurea |
| 25 | N-[4-(3-amino-1,2-benzisoxazol-4-yl)phenyl]-N′-(4-methylphenyl)urea |
| 26 | N-[4-(3-amino-1,2-benzisoxazol-4-yl)phenyl]-N′-(3-cyanophenyl)urea |
| 27 | N-[4-(3-amino-1,2-benzisoxazol-4-yl)phenyl]-N′-[4-fluoro-3- |
| | (trifluoromethyl)phenyl]urea |
| 28 | N-[4-(3-amino-1,2-benzisoxazol-4-yl)phenyl]-N′-(3-bromophenyl)urea |
| 29 | N-[4-(3-amino-1,2-benzisoxazol-4-yl)phenyl]-N′-(3-chlorophenyl)urea |
| 30 | N-[4-(3-amino-1,2-benzisoxazol-4-yl)phenyl]-N′-(3-ethylphenyl)urea |
| 31 | N-[4-(3-amino-1,2-benzisoxazol-4-yl)phenyl]-N′-[4-(trifluoromethyl)phenyl]urea |
| 32 | N-[4-(3-amino-1,2-benzisoxazol-4-yl)phenyl]-N′-(3-fluoro-4-methylphenyl)urea |
| 33 | N-[4-(3-amino-1,2-benzisoxazol-4-yl)phenyl]-N′-(3-fluorophenyl)urea |
| 34 | N-[4-(3-amino-1,2-benzisoxazol-4-yl)phenyl]-N′-(3,5-difluorophenyl)urea |
| 35 | N-[4-(3-amino-1,2-benzisoxazol-4-yl)phenyl]-N′-(3-methoxyphenyl)urea |
| 36 | N-[4-(3-amino-1,2-benzisoxazol-4-yl)phenyl]-N′-(4-methoxyphenyl)urea |
| 37 | N-[4-(3-amino-1,2-benzisoxazol-4-yl)phenyl]urea |
| 38 | N-[4-(3-amino-1,2-benzisoxazol-4-yl)phenyl]-N′-(3-nitrophenyl)urea |
| 39 | N-[4-(3-amino-1,2-benzisoxazol-4-yl)phenyl]-N′-(4-fluorophenyl)urea |
| 40 | N-[4-(3-amino-1,2-benzisoxazol-4-yl)phenyl]-N′-(2-fluorophenyl)urea |
| 41 | N-[4-(3-amino-1,2-benzisoxazol-4-yl)phenyl]-N′-(3-chloro-4-fluorophenyl)urea |
| 42 | N-[4-(3-amino-1,2-benzisoxazol-4-yl)phenyl]-N′-(3-chloro-4-methoxyphenyl)urea |
| 43 | N-[4-(3-amino-1,2-benzisoxazol-4-yl)phenyl]-N′-[4-(dimethylamino)phenyl]urea |
| 44 | N-[4-(3-amino-1,2-benzisoxazol-4-yl)phenyl]-N′-[4-(trifluoromethoxy)phenyl]urea |
| 45 | N-[4-(3-amino-1,2-benzisoxazol-4-yl)phenyl]-N′-[2-(trifluoromethoxy)phenyl]urea |
| 46 | N-[4-(3-amino-1,2-benzisoxazol-4-yl)phenyl]-N′-[3,5-bis(trifluoromethyl)phenyl]urea |
| 47 | N-[4-(3-amino-1,2-benzisoxazol-4-yl)phenyl]-N′-(3-chloro-4-methylphenyl)urea |
| 48 | N-[4-(3-amino-7-methoxy-1,2-benzisoxazol-4-yl)phenyl]-N′-[3,5- |
| | bis(trifluoromethyl)phenyl]urea |
| 49 | N-[4-(3-amino-7-methoxy-1,2-benzisoxazol-4-yl)phenyl]-N′-[4- |
| | (trifluoromethoxy)phenyl]urea |
| 50 | N-[4-(3-amino-7-methoxy-1,2-benzisoxazol-4-yl)phenyl]-N′-(3-fluorophenyl)urea |
| 51 | N-[4-(3-amino-7-methoxy-1,2-benzisoxazol-4-yl)phenyl]-N′-(3-methoxyphenyl)urea |
| 52 | N-[4-(3-amino-7-methoxy-1,2-benzisoxazol-4-yl)phenyl]-N′-(3,5-difluorophenyl)urea |
| 53 | N-[4-(3-amino-7-methoxy-1,2-benzisoxazol-4-yl)phenyl]-N′-(4-methylphenyl)urea |
| 54 | N-[4-(3-amino-7-methoxy-1,2-benzisoxazol-4-yl)phenyl]-N′-(3-bromophenyl)urea |
| 55 | N-[4-(3-amino-7-methoxy-1,2-benzisoxazol-4-yl)phenyl]-N′-(3,5-dimethylphenyl)urea |
| 56 | N-[4-(3-amino-7-methoxy-1,2-benzisoxazol-4-yl)phenyl]-N′-[4- |
| | (dimethylamino)phenyl]urea |
| 57 | N-[4-(3-amino-7-methyl-1,2-benzisoxazol-4-yl)phenyl]-N′-(3-methylphenyl)urea |
| 58 | N-[4-(3-amino-7-methyl-1,2-benzisoxazol-4-yl)phenyl]-N′-(3-chlorophenyl)urea |
| 59 | N-[4-(3-amino-7-methyl-1,2-benzisoxazol-4-yl)phenyl]-N′-(2-fluoro-5- |
| | methylphenyl)urea |
| 60 | N-[4-(3-amino-7-methyl-1,2-benzisoxazol-4-yl)phenyl]-N′-[2-fluoro-5- |
| | (trifluoromethyl)phenyl]urea |
| 61 | N-[4-(3-amino-7-methyl-1,2-benzisoxazol-4-yl)phenyl]-N′-[3- |
| | (trifluoromethyl)phenyl]urea |
| 62 | N-[4-(3-amino-7-methyl-1,2-benzisoxazol-4-yl)phenyl]-N′-(3,5-dimethylphenyl)urea |
| 63 | N-[4-(3-amino-7-methyl-1,2-benzisoxazol-4-yl)phenyl]-N′-(3-ethylphenyl)urea |
| 64 | N-[4-(3-amino-7-methyl-1,2-benzisoxazol-4-yl)phenyl]-N′-(4-methylphenyl)urea |
| 65 | N-[4-(3-amino-7-methyl-1,2-benzisoxazol-4-yl)phenyl]-N′-[4- |
| | (trifluoromethoxy)phenyl]urea |
| 66 | N-[4-(3-amino-7-methyl-1,2-benzisoxazol-4-yl)phenyl]-N′-(3-fluoro-4- |
| | methylphenyl)urea |
| 67 | N-[4-(3-amino-7-methyl-1,2-benzisoxazol-4-yl)phenyl]-N′-(3-methoxyphenyl)urea |
| 68 | N-[4-(3-amino-7-methyl-1,2-benzisoxazol-4-yl)phenyl]-N′-phenylurea |
| 69 | N-[4-(3-amino-7-methyl-1,2-benzisoxazol-4-yl)phenyl]-N′-[3,5- |
| | bis(trifluoromethyl)phenyl]urea |
| 70 | N-[4-(3-amino-7-methyl-1,2-benzisoxazol-4-yl)phenyl]-N′-(3-bromophenyl)urea |
| 71 | N-[4-(3-amino-7-methyl-1,2-benzisoxazol-4-yl)phenyl]-N′-(3-fluorophenyl)urea |
| 72 | N-[4-(3-amino-7-methoxy-1,2-benzisoxazol-4-yl)phenyl]-N′-[4-fluoro-3- |
| | (trifluoromethyl)phenyl]urea |
| 73 | N-[4-(3-amino-7-methoxy-1,2-benzisoxazol-4-yl)phenyl]-N′-(4-fluoro-3- |
| | methylphenyl)urea |
| 74 | N-[4-(3-amino-7-fluoro-1,2-benzisoxazol-4-yl)phenyl]-N′-[3- |
| | (trifluoromethyl)phenyl]urea |
| 75 | N-[4-(3-amino-7-fluoro-1,2-benzisoxazol-4-yl)phenyl]-N′-(3-chlorophenyl)urea |
| 76 | N-[4-(3-amino-7-fluoro-1,2-benzisoxazol-4-yl)phenyl]-N′-[4-fluoro-3- |
| | (trifluoromethyl)phenyl]urea |
| 77 | N-[4-(3-amino-7-fluoro-1,2-benzisoxazol-4-yl)phenyl]-N′-(3-methylphenyl)urea |
| 78 | N-[4-(3-amino-7-fluoro-1,2-benzisoxazol-4-yl)phenyl]-N′-[2-fluoro-5- |
| | (trifluoromethyl)phenyl]urea |
| 79 | N-[4-(3-amino-7-fluoro-1,2-benzisoxazol-4-yl)phenyl]-N′-(2-fluoro-5- |
| | methylphenyl)urea |
| 80 | N-{4-[3-amino-7-(trifluoromethoxy)-1,2-benzisoxazol-4-yl]phenyl}-N′-[2-fluoro-5- |
| | (trifluoromethyl)phenyl]urea |
| 81 | N-{4-[3-amino-7-(trifluoromethoxy)-1,2-benzisoxazol-4-yl]phenyl}-N′-[3- |
| | (trifluoromethyl)phenyl]urea |
| 82 | N-{4-[3-amino-7-(trifluoromethoxy)-1,2-benzisoxazol-4-yl]phenyl}-N′-(2-fluoro-5- |
| | methylphenyl)urea |
| 83 | N-{4-[3-amino-7-(trifluoromethoxy)-1,2-benzisoxazol-4-yl]phenyl}-N′-(3- |
| | chlorophenyl)urea |
| 84 | N-{4-[3-amino-7-(trifluoromethoxy)-1,2-benzisoxazol-4-yl]phenyl}-N′-(3- |
| | bromophenyl)urea |
| 85 | N-{4-[3-amino-7-(trifluoromethoxy)-1,2-benzisoxazol-4-yl]phenyl}-N′-[4-fluoro-3- |
| | (trifluoromethyl)phenyl]urea |
| 86 | N-[4-[3-amino-1H-indazol-4-yl]phenyl]-N′-(2-fluoro-5-methylphenyl)urea |
| 87 | N-{4-[3-amino-7-(trifluoromethoxy)-1,2-benzisoxazol-4-yl]phenyl}-N′-(4-fluoro-3- |
| | methylphenyl)urea |
| 88 | N-[4-(3-amino-1H-indazol-4-yl)phenyl]-N′-(3-methylphenyl)urea |
| 89 | N-[4-(3-amino-1H-indazol-4-yl)phenyl]-N′-(3,5-dimethoxyphenyl)urea |
| 90 | N-[4-(3-amino-1H-indazol-4-yl)phenyl]-N′-(3-chlorophenyl)urea |
| 91 | N-[4-(3-amino-1H-indazol-4-yl)phenyl]-N′-[3-(trifluoromethyl)phenyl]urea |
| 92 | N-[4-(3-amino-1H-indazol-4-yl)phenyl]-N′-[2-fluoro-5-(trifluormethyl)phenyl]urea |
| 93 | N-[4-(3-amino-1H-indazol-4-yl)phenyl]-N′-(3-bromophenyl)urea |
| 94 | N-[4-(3-amino-1H-indazol-4-yl)phenyl]-N′-(3-bromo-4-methylphenyl)urea |
| 95 | N-[4-(3-amino-1H-indazol-4-yl)phenyl]-N′-(3-ethylphenyl)urea |
| 96 | N-[4-(3-amino-1H-indazol-4-yl)phenyl]-N′-phenylurea |
| 97 | N-[4-(3-amino-1H-indazol-4-yl)phenyl]-N′-(3-fluoro-4-methylphenyl)urea |
| 98 | N-[4-(3-amino-1H-indazol-4-yl)phenyl]-N′-(2-fluorophenyl)urea |
| 99 | N-[4-(3-amino-1H-indazol-4-yl)phenyl]-N′-(4-fluorophenyl)urea |
| 100 | N-[4-(3-amino-1H-indazol-4-yl)phenyl]-N′-(3-fluorophenyl)urea |
| 101 | N-[4-(3-amino-1H-indazol-4-yl)phenyl]-N′-(3-hydroxyphenyl)urea |
| 102 | N-[4-(3-amino-1H-indazol-4-yl)phenyl]-N′-(3-methylphenyl)urea |
| 103 | N-[4-(3-amino-1H-indazol-4-yl)-2-fluorophenyl]-N′-(2-fluoro-5-methylphenyl)urea |
| 104 | N-[4-(3-amino-1H-indazol-4-yl)phenyl]-N′-[4-fluoro-3-(trifluoromethyl)phenyl]urea |
| 105 | N-[4-(3-amino-1H-indazol-4-yl)phenyl]-N′-[2-fluoro-3-(trifluoromethyl)phenyl]urea |
| 106 | N-[4-(3-amino-1H-indazol-4-yl)phenyl]-N′-(4-bromo-2-fluorophenyl)urea |
| 107 | N-[4-(3-amino-1H-indazol-4-yl)phenyl]-N′-(5-fluoro-2-methylphenyl)urea |
| 108 | N-[4-(3-amino-1H-indazol-4-yl)phenyl]-N′-(4-fluoro-3-methylphenyl)urea |
| 109 | N-[4-(3-amino-1H-indazol-4-yl)phenyl]-N′-[2-fluoro-5-(hydroxymethyl)phenyl]urea |
| 110 | 3-[({[4-(3-amino-1H-indazol-4-yl)phenyl]amino}carbonyl)amino]-4-fluorobenzoic |
| | acid |
| 111 | Methyl 3-[({[4-(3-amino-1H-indazol-4-yl)phenyl]amino}carbonyl)amino]-4- |
| | fluorobenzoate |

Preferred RTKi compounds for use in the methods of the invention include Compounds 86 and 88-111. The most preferred RTKi compound for use in the methods of the invention is Compound 86.

Vascular growth in the retina leads to visual degeneration culminating in blindness. Vascular endothelial growth factor (VEGF) accounts for most of the angiogenic activity produced in or near the retina in diabetic retinopathy. Ocular VEGF mRNA and protein are elevated by conditions such as retinal vein occlusion in primates and decreased pO₂ levels in mice that lead to neovascularization. Intraocular injections of either anti-VEGF monoclonal antibodies or VEGF receptor immunofusions inhibit ocular neovascularization in rodent and primate models. Regardless of the cause of induction of VEGF in human diabetic retinopathy, inhibition of ocular VEGF is useful in treating the disease.

Thus, it is further contemplated that compounds targeting VEGF receptors would be useful in the methods disclosed herein for preventing or inhibiting inflammation resulting from surgical procedures. Acceptable anti-VEGF compounds for use in the methods of the invention include any molecule that binds directly to VEGF and prevents ligand-receptor interaction (i.e., Macugen® (pegaptanib), Lucentis® (ranibizumab), Avastin® (bevacizumab), VEGF Trap) or any agent known to down-regulate VEGF production (i.e., siRNA molecules Cand5, Sima-027), directly or indirectly. Other known anti-angiogenic agents, such as anecortave acetate, anecortave desacetate, corticosteroids, HIF-1 inhibitors (e.g., rapamycin and its analogs) may also be used in the compositions and methods of the invention.

The models described in the examples below can be used to identify additional effective RTK inhibitor or anti-VEGF compounds for potential use in the methods of the invention, or to select preferred compounds from those identified via receptor binding assays that are well known to the skilled artisan. For example, a test compound may be evaluated in the rat OIR model described in Example 1, the mouse laser model described in Example 3, the rat VEGF model described in Example 6, and the diabetic rat model described in Example 7. Potential RTK inhibitors or anti-VEGF compounds (test compounds) for use in the methods of the invention, preferably provide:
>75% inhibition of preretinal NV in the rat OIR model following a single intravitreal injection of ≦3% solution or suspension, or oral gavage with a solution or suspension ≦30 mg/kg/d.
>70% inhibition of choroidal NV in the mouse laser model following a single intravitreal injection of ≦3% solution or suspension, or oral gavage with a solution or suspension <30 mg/kg/d.
>50% inhibition of retinal vascular permeability in rat VEGF model following a single intravitreal injection of ≦3% solution or suspension, or oral gavage with a solution or suspension <30 mg/kg/d.
>75% inhibition of retinal vascular permeability in the STZ-induced diabetic rat model following a single intravitreal injection of ≦3% solution or suspension, or oral gavage with a solution or suspension <30 mg/kg/d.

Compounds that are able to achieve activity in the categories described above are preferred agents with potential clinical utility. More preferred agents exhibit >25% regression of choroidal NV in the mouse laser model following a single intravitreal injection of ≦3% solution or suspension, or 50% regression with oral gavage with a solution or suspension <30 mg/kg/d.

The compositions of the invention will generally be administered to a patient during a surgical procedure in order to prevent or inhibit the occurrence of inflammation resulting from the trauma associated with the surgery. It may also be useful to administer the compositions before and/or after the surgical procedure as well. It is contemplated that the compositions of the invention may be useful in a treatment regimen after surgery in order to prevent inflammation from occurring or to limit the severity of the inflammation.

The compounds of the present invention may preferably be formulated into a variety of topically administrable ophthalmic compositions, such as solutions, suspensions, gels or ointments. For administration during surgery, the compositions are generally administered in the form of an irrigating solution. When administered after surgery, the compounds are more preferably administered in a suspension as eye drops; however, formulations wherein the final specialty form is a gel or ointment can also be employed and formulated according to conventional technology. The preferred suspensions of the present invention will typically contain a RTK inhibitor or anti-VEGF compound in an amount from about 0.001 to about 4.0% (w/v) or 0.024 to 160 μg/Kg total body weight/day, preferably in an amount from about 0.01 to about 0.5% (w/v) or 0.24 to 20 μg/Kg/day, and most preferably in an amount of about 0.1% (w/v) or 4 μg/Kg/day. The preferred suspensions of the present invention are preferably administered three times a day.

The compositions of the present invention may also contain conventionally employed ophthalmic adjuvants. Examples of such adjuvants include preservatives, such as thimerosal, chlorobutanol, benzalkonium chloride, Onamer M, or chlorhexidine; buffering agents, such as phosphates, borates, carbonates and citrates; and thickening agents, such as, high molecular weight carboxy vinyl polymers, such as, the ones sold under the name of Carbopol, which is a trademark of the B.F. Goodrich Chemical Company, hydroxyethylcellulose, and polyvinyl alcohol.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Prevention of Preretinal Neovascularization Following Intravitreal Delivery of the Receptor Kinase Tyrosine Inhibitor (RTKi), Compound 86, in the Rat Model of Oxygen-Induced Retinopathy

METHODS. Pregnant Sprague-Dawley rats were received at 14 days gestation and subsequently gave birth on Day 22±1 of gestation. Immediately following parturition, pups were pooled and randomized into separate litters (n=17 pups/litter), placed into separate shoebox cages inside oxygen delivery chamber, and subjected to an oxygen-exposure profile from Day 0-14 postpartum. Litters were then placed into room air from Day 14/0 through Day 14/6 (days 14-20 postpartum). Additionally on Day 14/0, each pup was randomly assigned as an oxygen-exposed control or into various treatment groups. For those randomized into an injection treatment group: one eye received a 5 μl intravitreal injection of 0.1%, 0.3%, 0.6%, or 1% RTKi and the contralateral eye received a 5 μl intravitreal injection of vehicle. At Day 14/6 (20 days postpartum), all animals in both studies were euthanized.

Immediately following euthanasia, retinas from all rat pups were harvested, fixed in 10% neutral buffered formalin for 24 hours, subjected to ADPase staining, and fixed onto slides as whole mounts. Digital images were acquired from each retinal flat mount that was adequately prepared. Computerized image analysis was used to obtain a NV clockhour score from each readable sample. Each clockhour out of 12 total per retina was assessed for the presence or absence of preretinal NV. Statistical comparisons using median scores for NV clockhours from each treatment group were utilized in nonparametric analyses. Each noninjected pup represented one NV score by taking the average value of both eyes and was used in comparisons against each dosage group. Because the pups were randomly assigned and no difference was observed between oxygen-exposed control pups from all litters, the NV scores were combined for all treatment groups. P≦0.05 was considered statistically significant.

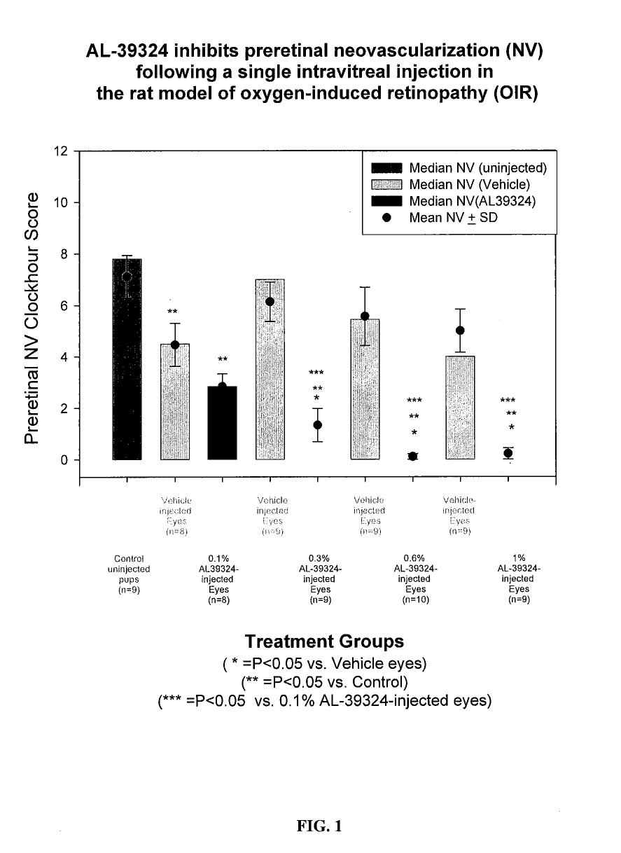

RESULTS. Local administration of RTKi provided potent anti-angiogenic efficacy against preretinal neovascularization, where 100% inhibition of preretinal NV was observed between 0.3%-1% suspensions. An overall statistical difference was demonstrated between treatment groups (Kiruskal-Wallis one-way ANOVA test: P<0.001) (FIG. 1). Eyes treated with 0.3-1% RTKi exhibited significant inhibition of preretinal NV as compared to vehicle-injected injected and control, noninjected eyes (Table 2). Efficacy was not observed in 0.1% treated eyes.

TABLE 2

| | % Inhibition | | | | Median | |
|---|---|---|---|---|---|---|
| | (vs. vehicle- | | Median | NV | NV | NV Range |
| Treatment | injected eye) | P value | NV | Range | (Vehicle) | (Vehicle) |
| Untreated control | | | 7.8 | 3.2-10.9 | | |
| 0.1% RTKi | 37 | 0.161 | 2.835 | 1.1-5.3 | 4.5 | 1.1-8.4 |
| 0.3% RTKi | 100 | 0.002 | 0 | 0-5 | 7 | 2-8.72 |
| 0.6% RTKi | 100 | <0.001 | 0 | 0-1.1 | 5.45 | 2-10.9 |
| 1% RTKi | 100 | <0.001 | 0 | 0-2 | 4 | 1-8 |

### EXAMPLE 2

### Systemic Administration of RTKi (Compound 86) Potently Prevents Preretinal Neovascularization in the Rat OIR Model.

METHODS. Pregnant Sprague-Dawley rats were received at 14 days gestation and subsequently gave birth on Day 22±1 of gestation. Immediately following parturition, pups were pooled and randomized into separate litters (n=17 pups/litter), placed into separate shoebox cages inside oxygen delivery chamber, and subjected to an oxygen-exposure profile from Day 0 to Day 14 postpartum. Litters were then placed into room air from Day 14/0 through Day 14/6 (days 14-20 postpartum). Additionally on Day 14/0, each pup was randomly assigned as oxygen-exposed controls, vehicle treated, or drug-treated at 1.5, 5, 10 mg/kg, p.o., BID. At Day 14/6 (20 days postpartum), all animals in both studies were euthanized and retina whole mounts were prepared as described in Example 1 above.

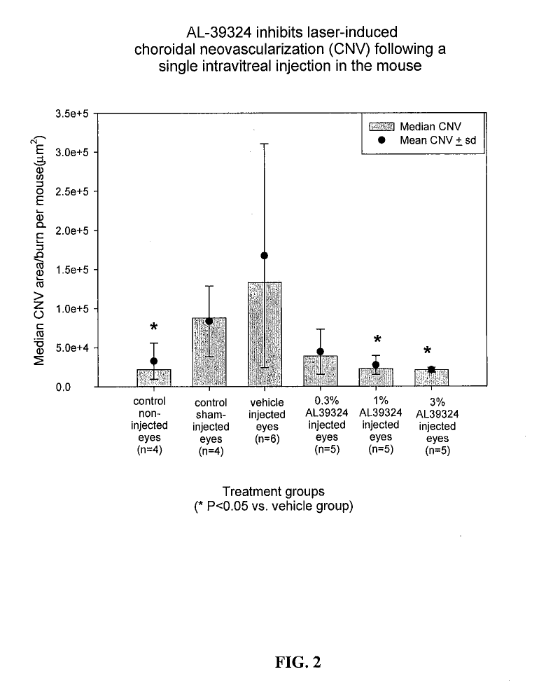

RESULTS. Systemic administration of RTKi provided potent efficacy in the rat OIR model, where 20 mg/kg/day p.o. provided complete inhibition of preretinal NV. An overall statistical difference was demonstrated between treatment groups and non-treated controls (Kiruskal-Wallis one-way ANOVA test: P<0.001) (FIG. 2, Table 3). Pups receiving 10 and 20 mg/kg/day p.o. demonstrated significant inhibition of preretinal NV as compared to vehicle-treated pups, where the highest dose provided complete inhibition (Mann-Whitney rank sum test: P=0.005 and P<0.001). Pups receiving 3 mg/kg/day p.o. did not have a significant decrease in NV.

TABLE 3

| | % Inhibition | | | |
|---|---|---|---|---|
| | (vs vehicle- | | | |
| | injected | P | Median | NV |
| Treatment | eye) | value | NV | Range |
| Untreated control | | 0.427 | 5.885 | 2.5-10.5 |
| PEG 400 (vehicle) | | | 7.4 | 3.5-9.5 |
| 3 mg/kg RTKi in PEG 400 | −1.4 | 0.91 | 7.5 | 3.8-9.8 |
| 10 mg/kg RTKi in PEG 400 | 86.4 | 0.001 | 1.105 | 0-8 |
| 20 mg/kg RTKi in PEG 400 | 100 | <0.001 | 0 | 0 |

### EXAMPLE 3

### Prevention of Laser-Induced Choroidal Neovascularization (CNV) Following a Intravitreal Delivery of the Receptor Kinase Tyrosine Inhibitor (RTKi), Compound 86, in the Mouse.

METHODS. CNV was generated by laser-induced rupture of Bruch's membrane. Briefly, 4 to 5 week old male C57BL/6J mice were anesthetized using intraperitoneal administration of ketamine hydrochloride (100 mg/kg) and xylazine (5 mg/kg) and the pupils of both eyes dilated with topical ocular instillation of 1% tropicamide and 2.5% Mydfin®. One drop of topical cellulose (Gonioscopic®) was used to lubricate the cornea. A hand-held cover slip was applied to the cornea and used as a contact lens to aid visualization of the fundus. Three to four retinal burns were placed in randomly assigned eye (right or left eye for each mouse) using the Alcon 532 nm EyeLite laser with a slit lamp delivery system. The laser burns were used to generate a rupture in Bruch's membrane, which was indicated ophthalmoscopically by the formation of a bubble under the retina. Only mice with laser burns that produced three bubbles per eye were included in the study. Burns were typically placed at the 3, 6, 9 or 12 o'clock positions in the posterior pole of the retina, avoiding the branch retinal arteries and veins.

Each mouse was randomly assigned into one of the following treatment groups: noninjected controls, sham-injected controls, vehicle-injected mice, or one of three RTKi-injected groups. Control mice received laser photocoagulation in both eyes, where one eye received a sham injection, i.e. a pars plana needle puncture. For intravitreal-injected animals, one laser-treated eye received a 5 ul intravitreal injection of 0%, 0.3%, 1%, or 3% RTKI. The intravitreal injection was performed immediately after laser photocoagulation. At 14 days post-laser, all mice were anesthetized and systemically perfused with fluorescein-labeled dextran. Eyes were then harvested and prepared as choroidal flat mounts with the RPE side oriented towards the observer. All choroidal flat mounts were examined using a fluorescent microscope. Digital images of the CNV were captured, where the CNV was identified as areas of hyperfluorescence within the pigmented background. Computerized image analysis was used to delineate and measure the two dimensional area of the hyperfluorescent CNV per lesion (um²) for the outcome measurement. The median CNV area/burn per mouse per treatment group or the mean CNV area/burn per treatment group was used for statistical analysis depending on the normality of data distribution; P≦0.05 was considered significant.

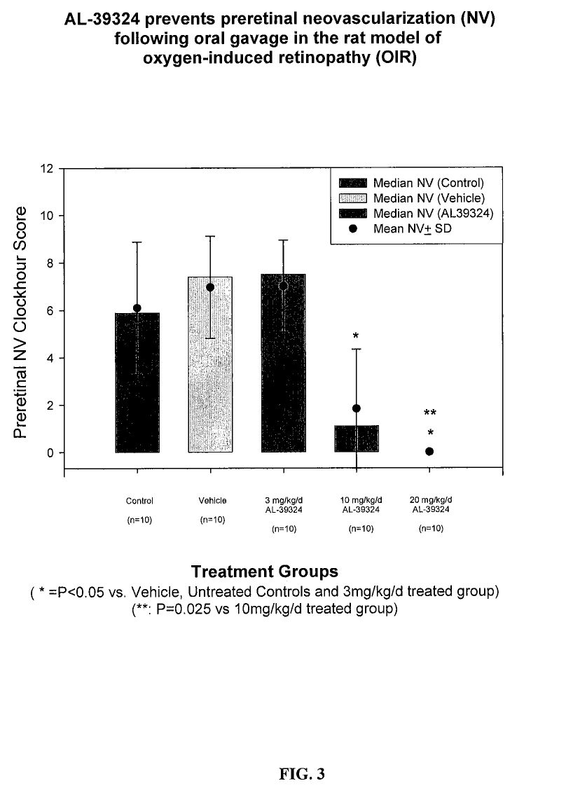

RESULTS. Local administration of RTKi provided potent antiangiogenic efficacy in a mouse model of laser-induced CNV. An overall significant difference between treatment groups was established with a Kiruskal-Wallis one way ANOVA (P=0.015) (FIG. 3). Moreover, eyes injected with 1% RTKi (⇓84.1%) and 3% RTKi (⇓83.0%) showed significant inhibition of CNV as compared to vehicle-injected eyes (Mann-Whitney rank sum tests; P=0.004, and P=0.017, respectively). A marginal statistical difference was found between eyes injected with 0.3% RTKi and vehicle injected eyes (P=0.082).

The median and mean±s.d. CNV area/burn per mouse in control groups with no injection was 21721 um² and 32612±23131 um²(n=4 mice), and with sham injection was 87854 um² and 83524±45144 um² (n=4 mice). The median and mean±s.d. CNV area/burn per mouse in vehicle-treated mice was 133014 um² and 167330±143201 um² (n=6 mice). The median/mean±s.d. in the 0.3%, 1% and 3% RTKi treated groups were 38891 um² and 44283±28886 um² (n=5 mice); 21122 um² and 21036±3100 um² (n=5 mice); 22665 um² and 27288±12109 um² (n=5 mice), respectively.

### EXAMPLE 4

### Intravitreal Delivery of the RTKi, Compound 86, Induces Regression of Existing Laser-Induced Choroidal Neovascularization (CNV) in the Mouse

METHODS. CNV was generated by laser-induced rupture of Bruch's membrane as described above in Example 3. Each mouse was randomly assigned to one of the following treatment groups: noninjected controls, sham-injected controls, vehicle-injected mice, RTKi injected groups. Control mice received laser photocoagulation in both eyes, where one eye received a sham injection, i.e. a pars plana needle puncture. For intravitreal-injected animals, one laser-treated eye received a 5 μl intravitreal injection of 0%, 1% or 3% RTKi or 2 μl 1% RTKi. All mice received laser photocagulation at day 0. For mice randomized to an injection group, a single intravitreal injection was performed at 7 days post-laser. Also at 7 days post-laser, several mice with no-injection were euthanized and their eyes used for controls. At 14 days post-laser, all remaining mice were euthanized and systemically perfused with fluorescein-labeled dextran. Eyes were then harvested and prepared as choroidal flat mounts with the RPE side oriented towards the observer. Choroidal flat mounts were analyzed as described above in Example 3.

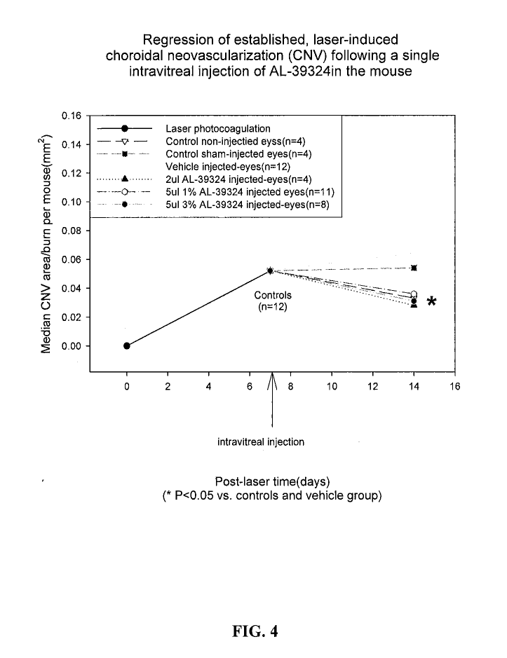

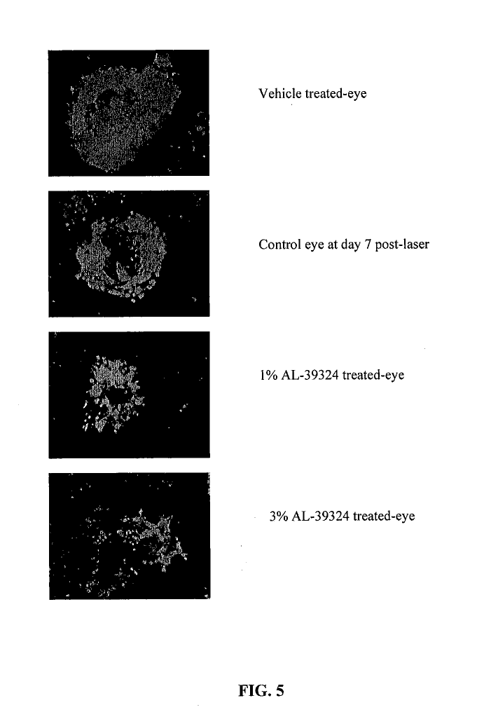

RESULTS. Local administration of RTKi caused regression of existing laser-induced CNV in the adult mouse. An overall significant difference between treatment groups was established with a Kiruskal-Wallis one way ANOVA (P=0.002) (FIG. 4). By 14 days following laser rupture of Bruch's membrane, the median CNV area in eyes injected with 2 μl 1% RTKi (⇓45.4%), 5 μl 1% RTKi (⇓29.7%), and 5 μl 3% RTKi (⇓41.0%) was significantly reduced when compared to the amount of CNV present at 7 days post-laser (Mann-Whitney rank sum tests; P=0.025, P=0.039 and P=0.012, respectively). Eyes injected with 2 μl 1% RTKi (⇓55.9%), 5 μl 1% RTKi (⇓43.7%), and 3% RTKi (⇓52.3%) showed significant inhibition of CNV as compared to vehicle-injected eyes at day 14 post-laser (Mann-Whitney rank sum tests; P=0.009, P=0.006, and 0.001, respectively). A gross reduction in CNV development was observed as a decrease in the hyperfluorescent area at the site of laser photocoagulation in 1% or 3% RTKi-injected eyes as compared to 1) control eyes at day 7 post-laser and 2) vehicle-injected eyes at day 14 post-laser (FIG. 5).

TABLE 4

| | Median | Mean | | N |
|---|---|---|---|---|
| | CNV(μm) | CNV(μm) | SE | (mice) |
| Control at day 7 | 51808 | 54452 | 5385 | 12 |
| Non-injected | 32881 | 34589 | 8413 | 4 |
| control at day 14 | | | | |
| Sham-injected | 54078 | 48594 | 8614 | 4 |
| control at day 14 | | | | |
| Vehicle | 64067 | 65932 | 5833 | 12 |
| 1% RTKi (2 μl) | 28268 | 30959 | 7287 | 4 |
| 1% RTKi (5 μl) | 36429 | 39178 | 5861 | 11 |
| 3% RTKi (5 μl) | 30560 | 35174 | 4110 | 8 |

### EXAMPLE 5

### Systemic Administration of the RTKi, Compound 86, Provides Dose-Dependent Inhibition and Regression of Laser-Induced Choroidal Neovascularization (CNV) in the Mouse.

METHODS. CNV was generated by laser-induced rupture of Bruch's membrane as described in Example 3 above. Mice were randomly assigned as oral gavage groups receiving 0, 3, 10, and 20 mg/kg/day RTKi. The mice received an oral gavage of 0, 1.5, 5, or 10 mg/kg twice per day and for 14 days post-laser. For the regression or intervention paradigm, mice were randomly assigned to groups receiving 0, 1.5, 5, or 10 mg/kg RTKi p.o. BID, (0, 3, 10, or 20 mg/kg/day) at day 7 after laser photocoagulation. Oral gavage dosing was continued twice per day for 14 days post-laser. Several mice were euthanized at day 7 post-laser and used for controls. At 14 days post-laser, all mice were anesthetized and systemically perfused with fluorescein-labeled dextran. Eyes were then harvested and prepared as choroidal flat mounts as described in Example 3 above.

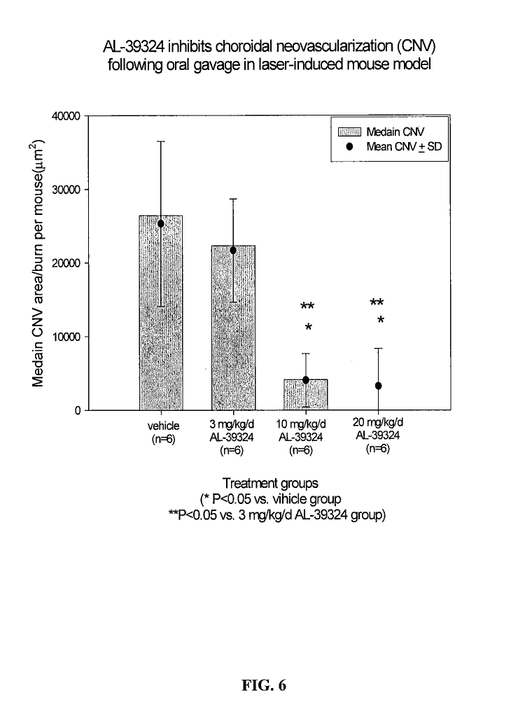

RESULTS. Systemic administration of RTKi provided potent and highly efficacious inhibition of laser-induced CNV, where mice treated 20 mg/kg/day showed complete inhibition of CNV development and significant regression of established CNV. In the prevention paradigm, an overall significant difference between treatment groups was established with a Kiruskal-Wallis one way ANOVA (P<0.001) (FIG. 6, Table 5a). Moreover, systemic delivery of 20 mg/kg/d RTKi provided complete inhibition of CNV (P<0.009) and the mice treated with 10 mg/kg/day showed an 84.3% inhibition of CNV (P<0.002). Mice treated with 3 mg/kg/day exhibited no significant inhibition (P<0.589), as compared to vehicle-injected eyes (Mann-Whitney rank sum tests).

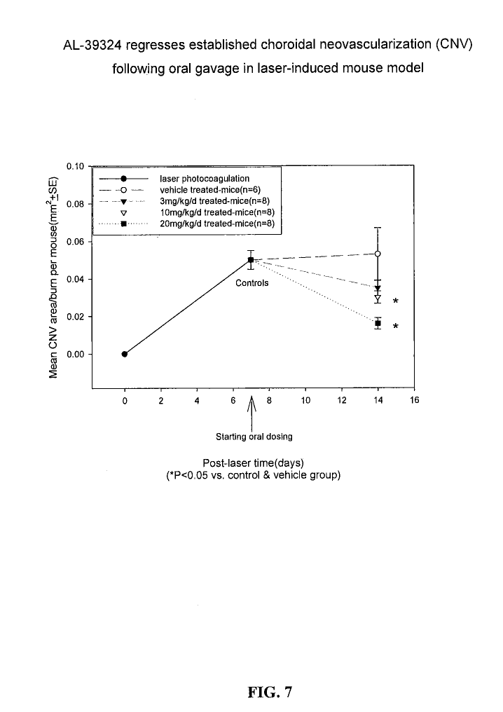

In the regression paradigm, an overall significant difference between treatment groups was established with a Kruskal-Wallis one-way ANOVA (P<0.001) (FIG. 7 & Table 5b). Mice treated with 20 mg/kg/day and 10 mg/kg/day exhibited significant regression of existing CNV by 68.0% and 41.8%, respectively, as compared to nontreated controls (Mann-Whitney Rank Sum Test, P<0.002 and P<0.011, respectively). Mice treated with 3 mg/kg/day did not show a significant regression of existing CNV (Mann-Whitney Rank Sum Test, P>0.065). No significant difference was found between the control and vehicle treated-groups (Mann-Whitney Rank Sum Test, P=0.792).

TABLE 5a

| | Median | Mean | | Mice |
|---|---|---|---|---|
| | CNV(μm²) | CNV(μm²) | SD | number |
| Vehicle | 26417 | 25316 | 11196 | 6 |
| 3 mg/kg/day RTKi | 22317 | 21670 | 7012 | 6 |
| 10 mg/kg/day RTKi | 4137 | 4046 | 3625 | 6 |
| 20 mg/kg/day RTKi | 0 | 3266 | 5079 | 6 |

TABLE 5b

| | Median | Mean | | Mice |
|---|---|---|---|---|
| Treatment | CNV(μm²) | CNV(μm²) | SD | number |
| Control | 47055 | 49665 | 11183 | 5 |
| Vehicle | 41362 | 52974 | 33403 | 6 |
| 3 mg/kg/day RTKi | 33967 | 35442 | 11807 | 8 |
| 10 mg/kg/day RTKi | 27389 | 29773 | 9514 | 8 |
| 20 mg/kg/day RTKi | 15036 | 15706 | 8301 | 8 |

### EXAMPLE 6

### Intravitreal Delivery of the RTKi, Compound 86, Inhibits VEGF-Induced Retinal Vascular Permeability in the Rat

METHODS. Adult Sprague-Dawley rats were anesthetized with intramuscular ketamine/xylazine and their pupils dilated with topical cycloplegics. Rats were randomly assigned to intravitreal injection groups of 0% 0.3%, 1.0%, and 3.0% RTKI and a positive control. Ten μl of each compound was intravitreally injected in each treatment eye (n=6 eyes per group). Three days following first intravitreal injection, all animals received an intravitreal injection of 10 μl 400 ng hr VEGF in both eyes. Twenty-four hours post-injection of VEGF, intravenous infusion of 3% Evans blue dye was performed in all animals, where 50 mg/kg of Evans blue dye was injected via the lateral tail vein during general anesthesia. After the dye had circulated for 90 minutes, the rats were euthanized. The rats were then systemically perfused with balanced salt solution, and then both eyes of each rat were immediately enucleated and the retinas harvested using a surgical microscope. After measurement of the retinal wet weight, the Evans blue dye was extracted by placing the retina in a 0.2 ml formamide (Sigma) and then the homogenized and ultracentrifuged. Blood samples were centrifuged and the plasma diluted 100 fold in formamide. For both retina and plasma samples, 60 μl of supernatant was used to measure the Evans blue dye absorbance (ABS) with at 620/740 nm. The blood-retinal barrier breakdown and subsequent retinal vascular permeability as measured by dye absorbance were calculated as means ±/-s.e.m. of net ABS/wet weight/plasma ABS. A two-tailed Student's t-test were used for pair wise comparisons between OS and OD eyes in each group. One way ANOVA was used to determine an overall difference between treatment means, where P≦0.05 was considered significant.

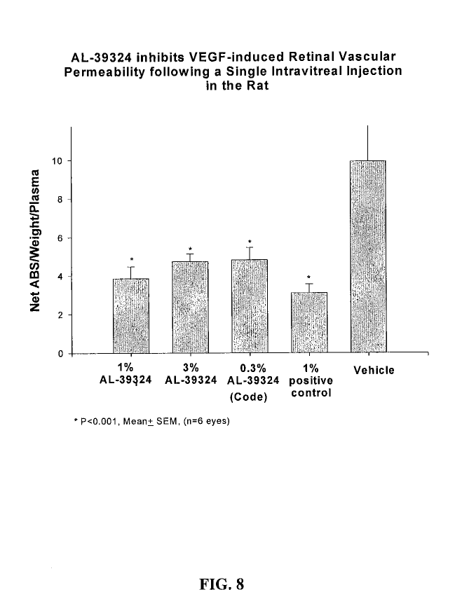

RESULTS. A single intravitreal injection of RTKi provided potent and efficacious inhibition of VEGF-induced retinal vascular permeability in the rat (FIG. 8). An overall statistical difference was demonstrated between treatment groups and vehicle controls (Student-Newman-Keuls one-way AVOVA test: P<0.001). Retinal vascular permeability was significantly decreased in eyes treated with RTK inhibitor as compared to vehicle-injected eyes: 0.3% RTKi (⇓50%), 1.0% RTKi (⇓61%), 3% RTKi (⇓53%), and positive control (⇓69%), respectively.

The mean ABS±s.e.m. in vehicle control group was 9.93±1.82. In drug treated group of 0.3% RTKI was 4.84±0.64; in 1.0% RTKI group was 3.87±0.62; in 3.0% RTKI group was 4.75±0.40 and in the positive control group was 3.11±0.46. There was no significant difference between drug treated groups.

### EXAMPLE 7

### Intravitreal Delivery of the RTKi, Compound 86, Inhibits VEGF-Induced Retinal Vascular Permeability in the Rat

METHODS. Diabetes was induced in male Long-Evans rats with 65 mg/kg streptozotocin (STZ) after an overnight fast. Upon confirmation of diabetes (blood glucose >250 mg/dl), treatment was initiated by oral gavage. Non-diabetic (NDM) and diabetic (DM) rats received oral gavage of either vehicle or RTK inhibitor at 1.5 or 5 mg/kg/d BID. After 2 weeks, jugular vein catheters were implanted 1 day prior to experimentation for the infusion of indicator dye. Retinal vascular permeability, RVP, was measured using Evan's blue albumin permeation (45 mg/kg) after a 2 hour circulation period.

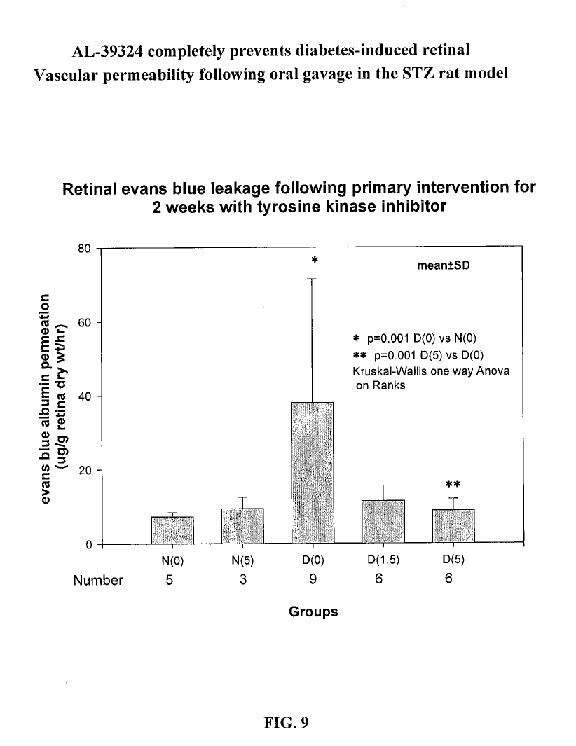

RESULTS. Treatment with the oral RTKi was well tolerated by both NDM and DM groups with no observed systemic or ERG side effects. Blood glucose levels and body weights were not different between DM control and DM treatment groups. Diabetes increased RVP (38.1±33.4 μl/g/hr, n=9) as compared with NDM control (7.3±2.5 μl/g/hr, n=5, p<0.001). RVP was significantly reduced in DM animals treated with RTKI at 1.5 mg/kg/d (11.4±4.1 μl/g/hr, n=6, p<0.05) and at 5 mg/kg/d (8.9±3.1 μl/g/hr, n=7, p<0.01) as compared to DM control (FIG. 9). RVP was unchanged in NDM treated at 5 mg/kg/d.

### EXAMPLE 8

The following examples of ophthalmic compositions typify the manner in which the invention may be practiced. The examples should be construed as illustrative, and not as a limitation upon the overall scope of the invention. The percentages are expressed on a weight/volume basis. “Active Agent” means a RTK inhibitor or anti-VEGF compound.

Formulation 1

| | |
|---|---|
| Active agent | 0.1% |
| Polysorbate 80 | 0.01% |
| Benzalkonium Chloride | 0.01% + 10% excess |
| Disodium EDTA | 0.1% |
| Monobasic Sodium Phosphate | 0.03% |
| Dibasic Sodium Phosphate | 0.1% |
| Sodium Chloride | q.s. 290-300 mOsm/Kg |
| pH adjustment with NaOH and/or HCl | pH 4.2-7.4 |
| Water | q.s. 100% |

Formulation 2

| | |
|---|---|
| Active Agent | 0.1% |
| Hydroxypropyl Methylcellulose | 0.5% |
| Polysorbate 80 | 0.01% |
| Benzalkonium Chloride | 0.01% + 5% excess |
| Disodium EDTA | 0.01% |
| Dibasic Sodium Phosphate | 0.2% |
| Sodium Chloride | q.s. 290-300 mOsm/Kg |
| pH adjustment with NaOH and/or HCl | pH 4.2-7.4 |
| Water | q.s. 100% |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

United States Patents

U.S. Pat. No. 4,045,576

U.S. Pat. No. 4,126,635

U.S. Pat. No. 4,254,146

U.S. Pat. No. 4,313,949

U.S. Pat. No. 4,503,073

U.S. Pat. No. 4,568,695

U.S. Pat. No. 4,683,242

U.S. Pat. No. 4,910,225

U.S. Pat. No. 5,475,034

U.S. Pat. No. 6.066,671

UK application no. 2071086A

UK application no. 2093027A

Other Publications
http://www.cellsignal.com/retail/
Adamis A P, Shima D T, Tolentino M J, et al. Inhibition of vascular endothelial growth factor prevents retinal ischemia-associated iris neovascularization in a nonhuman primate. Arch Ophthalmol. 1996;114:66-71.
Armstrong S A, Kung A L, Mabon M E, et al. Inhibition of FLT3 in MLL. Validation of a therapeutic target identified by gene expression based classification. Canc Cell. 2003;3:173-83.
Asahara T, Chen D, Takahashi T, et al. Tie2 receptor ligands, angiopoietin-1 and angiopoietin-2, modulate VEGF-induced postnatal neovascularization. Circ Res. 1998;83:233-40.
Benjamin Le, Hemo I, Keshet E. A plasticity window for blood vessel remodelling is defined by pericyte coverage of the preformed endothelial network and is regulated by PDGF-B and VEGF. Development. 1998;125:1591-8.
Bergers G, Song S, Meyer-Morse N, Bergsland E, Hanahan D. Benefits of targeting both pericytes and endothelial cells in the tumor vasculature with kinase inhibitors. J Clin Inv. 2003;111:1287-95.
Bilodeau M T, Fraley M E, Hartman G D. Kinase insert domain-containing receptor kinase inhibitors as anti-angiogenic agents. Expert Opin Investig Drugs. 2002;11(6):737-45.
Blume-Jensen P, Hunter T. Oncogenic kinase signalling. Nature. 2001;411:355-65.
Boyer S J. Small molecule inhibitors of KDR (VEGFR-2) kinase: An overview of structure activity relationships. Curr Top Med Chem. 2002;2:973-1000.
Campochiaro P A, the C99-PKC412-003 Study Group. Reduction of diabetic macular edema by oral administration of the kinase inhibitor PKC412. IOVS. 2004;45:922-31.
Carmeliet P, Rerreira V, Breier G, et al. Abnormal blood vessel development and lethality in embryos lacking a single VEGF allele. Nature. 1996;380:435-9.
Chen Y-S, Hackett S F, Schoenfeld C-L, Vinores M A, Vinores S A, Campochiaro P A. Localisation of vascular endothelial growth factor and its receptors to cells of vascular and avascular epiretinal membranes. Br J Ophthalmol. 1997;81:919-26.
Chomczynski P, Sacchi N. Single-step method of RNA isolation by acid guanidinium thiocyanate-phenol-chloroform extraction. Anal. Biochem. 1987;162:156-159.
Cousins S W, Espinosa-Heidmann D G, Csaky K G. Monocyte activation in patients with age-related macular degeneration—A biomarker of risk for choroidal neovascularization? Arch Ophthalmol. 2004;122(7):1013-8.
Csaky K G, Baffi J Z, Byrnes G A, et al. Recruitment of marrow-derived endothelial cells to experimental choroidal neovascularization by local expression of vascular endothelial growth factor. Exp Eye Res. 2004;78:1107-16.
Curtin M L, Frey R R, Heyman R, et al. Isoindolinone ureas: a novel class of KDR kinase inhibitors. Bioorg Med Chem Lett. 2004;14:4505-9.
De Vries C, Escobedo J A, Ueno H, Houck K, Ferrar N, Williams L T. The fms-like tyrosine kinase, a receptor for vascular endothelial growth factor. Science. 1992;255:989-91.
Dehmel U, Zaborski M, Meierhoff G, et al. Effects of FLT3 ligand on human leukemia cells. I. Proliferative response of myeloid leukemia cells. Leukemia. 1996;10:261-70.
Edelman J L, Castro M R. Quantitative image analysis of laser-induced choroidal neovascularization in rat. Exp Eye Res 2000;71:523-533.
Eriksson U, Alitalo K. VLGF receptor-1 stimulates stem-cell recruitment and new hope for angiogenesis therapies. Nat Med. 2002;8:775-7.
Espinosa-Heidmann D G, Caicedo A, Hernandez E P, Csaky K G, Cousins S W. Bone marrow-derived progenitor cells contribute to experimental choroidal neovascularization. IOVS. 2003;44(11):4914-19.
Eyetech Study Group. Preclinical and phase 1A clinical evaluation of an anti-VEGF pegylated aptamer (EYE001) for the treatment of exudative age-related macular degeneration. Retina. 2002;22:143-52.
Ferrara N, Davis-Smyth T. The biology of vascular endothelial growth factor. Endo Rev. 1997;18:4-25
Ferrara N, Gerber H P, LeCouter J. The biology of VLGF and its receptors. Nat Med. 2003;9:669-76.
Fong G H, Rossant J, Gertsenstein M, Breitman M L. Role of the Flt-1 receptor tyrosine kinase in regulating the assembly of vascular endothelium. Nature. 1995;376:66-70.
Fukumura D, Xavier R, Sugiura T, et al. Tumor induction of VEGF promoter activity in stromal cells. Cell. 1998;94:715-25.
George D, Platelet-derived growth factor receptors: A therapeutic target in solid tumors. Semin Oncol. 2001;28:27-33.
Grant M B, May W S, Caballero S, et al. Adult hematopoietic stem cells provide functional hemangioblast activity during retinal neovascularization. Nature Med. 2002;6(8):607-12.
Griffioen A W, Molema G. Angiogenesis: Potentials for pharmacologic intervention in the treatment of cancer, cardovascular diseases, and chronic inflammation. Pharm Rev. 2000;52:237-68.
Gschwind A, Fischer O M, Ullrich A. The discovery of receptor tyrosine kinases: targets for cancer therapy. Nat Rev. 2004;4:361-70.
Hackett S F, Ozaki H, Strauss R W, et al. Angiopoietin 2 expression in the retina: Upregulation during physiologic and pathologic neovascularization. J Cell Physiol. 2000;184:275-84.
Hammes H-P, Lin J, Wagner, et al. Angiopoietin-2 causes pericyte dropout in the normal retina: Evidence for involvement in diabetic retinopathy. Diabetes. 2004;53:1104-10.
Hanahan D. Signaling vascular morphogenesis and maintenance. Science. 1997;277:48-50.
Hartnett M E, Lappas A, Darland D, McColm J R, Lovejoy S, D'Amore P A. Retinal pigment epithelium and endothelial cell interaction causes retinal pigment epithelial barrier disfunction via a soluble VEGF-dependent mechanism. Exp Eye Res. 2003;77:593-9.
Heinrich M C, Blanke C D, Druker B J, Corless C L. Inhibition of KIT tyrosine kinase activity: A novel molecular approach to the treatment of KIT-positive malignancies. J Clin Oncol. 2002;20:1692-1703.
Hellström M, Kalén M, Lindahl P, Abramsson A, Betsholtz C. Role of PDGF-B and PDGFR-beta in recruitment of vascular smooth muscle cells and pericytes during embryonic blood vessel formation in the mouse. Development. 1999;126:3047-55.
Hunter T. Signaling—100 and beyond. Cell. 2000;100:113-127.
Inoue M, Hager J H, Ferrara N, Gerber H P, Hanahan D. VEGF-A has a critical, nonredundant role in angiogenic switching and pancreatic beta cell carcinogenesis. Cancer Cell. 2002;1:193-202.
Ishida S, Usui T, Yamashiro K, Kaji Y, Ahmed E, Carrasquillo K G, Amano S, Hida T, Oguchi Y, Adamis A P. VEGF₁₆₄ is proinflammatory in the diabetic retina. IOVS. 2003;44(5):2155-62.
Ishida S, Usui T, Yamashiro K, et al. VEGF₁₆₄ -mediated inflammation is required for pathological, but not physiological, ischemia-induced retinal neovascularization. J Exp Med. 2003;198(3):483-9.
Keck P J, Hauser S D, Krivi G, Sanzo K, Warren T, Feder J, Connolly. Vascular permeability factor, an endothelial cell mitogen related to PDGF. Science. 1989;246:1309-12.
Kiyoi H, Naoe T. FLT3 in human hematologic malignancies. Leukemia Lymphoma. 2002;43:1541-7.
Kottaridis P D, Gale R E, Linch D C. Flt3 mutations and leukaemia. Br J Haem. 2003;122:523-38.
Krishnan J, Kirkin V, Steffen A, et al. Differential in vivo and in vitro expression of vascular endothelial growth factor (VEGF)-C and VEGF-D in tumors and its relationship to lymphatic metastasis in immunocompetent rats. Cancer Res. 2003; 63:713-22.
Krzystolik M G, Afshari M A, Adamis A P, et al. Prevention of experimental choroidal neovascularization with intravitreal anti-vascular endothelial growth factor antibody fragment. Arch Ophthalmol. 2002;120:338-46.
Kvanta A, Algvere P V, Berglin L, Seregard S. Subfoveal fibrovascular membranes in age-related macular degeneration express vascular endothelial growth factor. IOVS. 1996;37(9):1929-34.
Kwak N, Okamoto N, Wood J M, Campochiaro P A. VEGF is major stimulator in model of choroidal neovascularization. IOVS. 200;41:3158-64.
Lawrence D S, Niu J. Protein kinase inhibitors: The tyrosine-specific protein kinases. Pharmacol Ther. 1998;77(2):81-114.
Leung D W, Cachianes G, Kuang W-J, Goeddel D V, Ferrara N. Vascular endothelial growth factor is a secreted angiogenic mitogen. Science. 1989;246:1306-9.
Levis M, Small D. FLT3: It does matter in leukemia. Leukemia. 2003;17:1738-52.
Lindahl P, Johansson B R, Leveen P, Betsholtz C. Pericyte loss and microaneurysm formation in PDGF-B-deficient mice. Science. 1997;277:242-5.
Lutty G A, McLeod D S, Merges C, Diggs A, Plouet J. Localization of vascular endothelial growth factor in human retina and choroid. Arch Ophthalmol. 1996;114:971-7.
Lyden D, Hattor K, Dias S, et al. Impaired recruitment of bone-marrow-derived endothelial and hematopoietic precursor cells blocks tumor angiogenesis and growth. Nat Med. 2001;7:1194-1201.
Manley P W, Furet P, Bold G. Anthranilic acid amides: A novel class of antiangiogenic VEGF receptor kinase inhibitors. J Med Chem. 2002;45:5687-93.
Manning G, Whyte D B, Martinez R, Hunter T, Sudarsanam S. The protein kinase complement of the human genome. Sciencel 2002;298:1912-34.
McMahon G. Presentation given at the 1^{st} International Symposium on Signal Transduction Modifiers in Cancer Therapy; Sep. 23, 2002. Amsterdam, NL.
Millauer B, Wizigmann-Voos S, Schnurch H, Martinez R, Moller N P, Risau W, Ullrich A. High affinity VEGF binding and developmental expression suggest Flk-1 as a major regulator of vasculogenesis and angiogenesis. Cell. 1993;72:835-46.
Miller J W, Adamis A P, Shima D T, et al. Vascular endothelial growth factor/vascular permeability factor is temporally and spatially correlated with ocular angiogenesis in a primate model. Am J Pathol. 1994;145(3)574-84.
Mudhar H S, Pollock R A, Wang C, Stiles C D, Richardson W D. PDGF and its receptors in the developing rodent retina and optic nerve. Development. 1993;118:539-52.
Murukata C, Kaneko M, Gessner G, et al. Mixed lineage kinase activity of indolocarbazole analogues. Bioorg Med Chem Let. 2002;12:147-50.
Nakao M, Yokota S, Iwai T, et al. Internal tandem duplication of the flt3 gene found in acute myeloid leukemia. Leukemia. 1996;10:1911-8.
Natali P G, Nicotra M R, Sures I, Santoro E, Bigotti A, Ullrich A. Expression of c-kit receptro in normal and transformed human nonlymphoid tisues. Cancer Res. 1992;52:6139-43.
Oh H, Takagi H, Suzuma K, Otani A, Matsumura M, Honda Y. Hypoxia and vascular endothelial growth factor selectively up-regulate angiopoietin-2 in bovine microvascular endothelial cells. J Bio Chem. 1999;274(22):15732-9.
Ohashi H, Takagi H, Koyama S, et al. Alterations in expression of angiopoietins and the Tie-2 receptor in the retina of streptozotocin induced diabetic rats. Mol Vis. 2004;10:608-17.
Ostman A, Heldin C H. Involvement of platelet-derived growth factor in disease: Development of specific antagonists. Adv Cancer Res. 2001;20:1-38.
Otani A, Takagi H, Oh H, Koyama S, Matsumura M, Honda Y. Expressions of angiopoietins and Tie2 in human choroidal neovascular membranes. IOVS. 1999;40(9)1912-20.
Ozaki H, Seo M-S, Ozaki K, et al. Blockade of vascular endothelial cell growth factor receptor signaling is sufficient to completely prevent retinal neovascularization. Am J Pathol. 200;156(2)697-707.
Pietras K, Rubin K, Sjoblom T, et al. Inhibition of PDGF receptor signaling in tumor stroma enhances antitumor effect of chemotherapy. Cancer Res. 2002;62:5476-84.
Ponten F, Ren Z, Nister M, Westermark B, Ponten J. Epithelial-stromal interactions in basal cell cancer: the PDGF system. J Inv Derm. 1994;102:304-9
Quinn T P, Peters K G, de Vries C, Ferrara N, Williams L T. Fetal liver kinase 1 is a receptor for vascular endothelial growth factor and is selectively expressed in vascular endothelium. Proc Natl Acad Sci. 1993;90:7533-7.
Rafii S, Lyden D, Benezra R, Hattori K, Heissig B. Vascular and haematopoietic stem cells: Novel targets for anti-angiogenesis therapy? Nat Rev Cancer. 2002;2:826-35.
Rak J W, St Croix B D, Kerbel R S. Consequences of angiogenesis for tumor progression, metastasis and cancer therapy. Anti-Cancer Drugs. 1995;6:3-18.
Reinmuth N, Liu W, Jung Y D, et al. Induction of VEGF in perivascular cells defines a potential paracrine mechanism for endothelial cell survival. FASEB J. 2001;15:1239-41.
Robinson D R, Wu Y M, Lin S F. The protein tyrosine kinase family of the human genome. Oncogene. 2000;19:5548-57.
Rosnet O, Buhring H J, deLapeyriere O, et al. Expression and signal transduction of the FLT3 tyrosine kinase receptor. Acta Haem. 1996;95:218-23.
Rosnet O, Schiff C, Pebusque M J, et al. Human FLT3/FLK2 gene: cDNA cloning and expression in hematopoietic cells. Blood. 1993;82:1110-9.
Saishin Y, Saishin Y, Takahashi K, Silva R L E, Hylton D, Rudge J S, Wiegand S J, Campochiaro P A. VEGF-TRAP_{R1R2} suppresses choroidal neovascularization and VEGF-induced breakdown of the blood-retinal barrier. J Cell Physiol. 2003;195:241-8.
Sarlos S, Rizkalla B, Moravski C J, Cao Z, Cooper M E, Wilkinson-Berka J L. Retinal angiogenesis is mediated by an interaction between the angiotensin type 2 receptor, VEGF, and angiopoietin. Am J Pathol. 2003;163(3):879-87.
Sawyers C L. Finding the next Gleevec: FLT3 targeted kinase inhibitor therapy for acute myeloid leukemia. Canc Cell. 2002;1:413-5.
Schlessinger J. Cell signaling by receptor tyrosine kinases. Cell. 2000;103:211-25.
Seo M S, Kwak N, Ozaki H, et al. Dramatic inhibition of retinal and choroidal neovascularization by oral administration of a kinase inhibitor. Am J Pathol. 199;154(6):1743-53.
Shaheen R M, Tseng W W, Davis D W, et al. Tyrosine kinase inhibition of multiple angiogenic growth factor receptors improves survival in mice bearing colon cancer liver metastases by inhibition of endothelial cell survival mechanisms. Canc Res. 2001;61:1464-8.
Shalaby F, Rossant J, Yamaguchi T P, Gertsenstein M, Wu X F, Breitman M L, Schuh. Failure of blood-island formation and vasculogenesis in Flk-1-defecient mice. Nature. 1995;376:62-66.
Shen W Y, Yu M J T, Barry C J, Constable I J, Rakoczy P E. Expression of cell adhesion molecules and vascular endothelial growth factor in experimental choroidal neovascularisation in the rat. Br J Opthalmol. 1998;82:1063-71.
Sherr C J, Rettenmier C W, Sacca R, Roussel M F, Look A T, Stanley E R. The c-fins proto-oncogene product is related to the receptor for the mononuclear phagocyte growth factor, CSF-1. Cell. 1985;41:665-76.
Shima D T, Adamis A P, Ferrara N, Yeo K-T, Yeo T-K, Allende R, Folkman J, D'Amore P A. Hypoxic induction of endothelial cell growth factors in retinal cells: Identification and characterization of vascular endothelial growth factor (VEGF) as the mitogen. Mol Med. 1995;1(2):182-93.
Skobe M, Fusenig N E. Tumorigenic conversion of immortal human keratinocytes through stromal cell activation. Proc Natl Acad Sci. 1998;95:1050-5.
Sorbera L A, Leeson P A, Bayés M. Ranibizumab. Drugs Future. 2003;28(6):541-5.
Stirewalt D L, Radich J P. The role of FLT3 in haematopoietic malignancies. Nat Rev Cancer. 2003;3:650-65.
Stone J, Itin A, Alon T, Pe'er J, Gnessin H, Chan-Ling T, Keshet E. Development of retinal vasculature is mediated by hypoxia-induced vascular endothelial growth factor (VEGF) expression by neuroglia. J Neurosci. 1995;15(7):4738-47.
Takagi H, Koyama S, Seike H, et al. Potential role of the angiopoietin/Tie2 system in ischemia-induced retinal neovascularization. IOVS. 2003;44(1):393-402.
Takahashi K, Saishin Y, Saishin Y, Mori K, Ando A, Yamamoto S, Oshima Y, Nambu H, Melia M B, Bingaman D P, Campochiaro P A. Topical nepafeac inhibits ocular neovascularization. IOVS. 2003;44(1):409-415.
Terman B I, Dougher-Vermazen M, Carrion M E, Dimitrov D, Armellino D C, Gospodarowicz D, Bohlen P. Identificatio of the KDR tyrosine kinase as a receptor for vascular endothelial cell growth factor. Biochem Biophys Res Comm. 1992;187:1579-86.
Tian Q, Frierson H F Jr, Krystal G W, Moskaluk C A. Activating c-kit gene mutations in human germ cell tumors. Am J Pathol. 1999;154:1643-7.
Tobe T, Okamoto N, Vinores M A, et al. Evolution of neovascularization in mice with overexpression of vascular endothelial growth factor in photoreceptors. Invest Ophthalmol Vis Sci 1998;39:180-188.
Tobe T, Ortega S, Luna L, et al. Targeted disruption of the FGF2 gene does not prevent choroidal neovascularization in a murine model. Am J Pathol 1998;153:1641-1646.
Tolentino M J, Miller J W, Gragoudas E S, et al. Vascular endothelial growth factor is sufficient to produce iris neovascularization and neovascular glaucoma in a nonhuman primate. Arch Ophthalmol. 1996;114:964-70.
Tolentino M J, Miller J W, Gragoudas E S, et al. Intravitreous injections of vascular endothelial growth factor produce retinal ischemia and microangiopathy in an adult primate. Ophthalmol. 1996;103:1820-8.
Traxler P, Bold G, Buchdunger E, Caravatti G, et al. Tyrosine kinase inhibitors: From rational design to clinical trials. Med Res Rev. 2001;21(6):499-512.
Turner A M, Zsebo K M, Martin F, Jacobsen F W, Bennett L C, Broudy V C. Nonhematopoietic tumor cell lines express stem cell factor and display c-kit receptors. Blood. 1992;80:374-81.
Unsoeld A S, Junker B, Mazitschek R, et al. Local injeciton of receptor tyrosine kinase inhibitor MAE 87 reduces retinal neovascularization in mice. Mol Vis. 2004;10:468-75.
Walsh et al., J. MED. CHEM. 33:2296-2304 (1990).
Waltenberger J, Claesson-Welsh L, Siegbahn A, Shibuya M, Heldin C H. Different signal transduction properties of KDR and Flt1, two receptors for vascular endothelial growth factor. J Bio Chem. 1994;269:26988-95.
Wang D, Huang H J, Kazlauskas A, Cavenee W K. Induction of vascular endothelial growth factor expression in endothelial cells by platelet-derived growth factor through the activation of phosphatidylinositol 3-kinase. Cancer Res. 1999;59:1464-72.
Werdich X Q, McCollum G W, Rajaratnam V S, Penn J S. Variable oxygen and retinal VEGF levels: correlation with incidence and severity of pathology in a rat model of oxygen-induced retinopathy. Exp Eye Res. 2004;79:623-30.
Wiesmann C, Fuh G, Christinger H W, EigenbrotC, Wells J A, de Vos, AM. Crystal structure at 1.7 Å resolution of VEGF in complex with domain-2 of the Flt-1 receptor. Cell. 1997;91:695-704.
Wilkinson-Berka J L, Babic S, De-Gooyer T, et al. Inhibition of platelet-derived growth factor promotes pericyte loss and angiogenesis in ischemic retinopathy. Am J Pathol. 2004;164(4):1263-73.
Witmer A N, Blaauwgeers H G, Weich H A, Alitalo K, Vrensen G F J M, Schlingemann R O. Altered expression patterns of VEGF receptors in human diabetic retina and in experimental VEGF-induced retinopathy in monkey. IOVS. 2002;43(3):849-57.
Yancopoulos G D, Davis S, Gale N W, Rudge J S, Wiegand S J, Holash J. Vascular-specific growth factors and blood vessel formation. Nature. 2000;407:242-8.

We claim:

1. A method for preventing inflammation and retinal edema resulting from an ocular surgical procedure, said method comprising administering to a patient undergoing surgery a therapeutically effective amount of a composition comprising an active agent selected from the group consisting of RTK inhibitors and anti-VEGF compounds, and a pharmaceutically acceptable carrier, wherein the RTK inhibitor blocks tyrosine autophosphorylation of VEGFR-1, VEGFR-2, VEGFR-3, Tie-2, PDGFR, c-KIT, Flt-3, and CSF-1R.

2. The method of claim 1, wherein the active agent is a RTK inhibitor.

3. The method of claim 2, wherein the RTK inhibitor has an IC₅₀ of from 0.1 nM to 250 nM for each of the receptors listed in claim 1.

4. The method of claim 2, wherein the RTK inhibitor blocks tyrosine autophosphorylation of Tie-2, PDGFR, and VEGF receptor 2 with an IC₅₀ of from 0.1 nM to 200 nM for each receptor.

5. The method of claim 3, wherein the RTK inhibitor has an IC₅₀ of from 0.1 nM to 100 nM for at least six of the receptor listed in claim 1.

6. The method of claim 5, wherein the RTK inhibitor has an IC₅₀ of from 0.1 nM to 10 nM for at least four of the receptors listed in claim 1.

7. The method of claim 2, wherein the RTK inhibitor blocks tyrosine autophosphorylation of VEGF receptor 2, VEGF receptor 1, PDGFR, and Tie-2.

8. The method of claim 7, wherein the RTK inhibitor has an IC₅₀ of from 0.1 nM to 200 nM for each of the receptors listed in claim 7.

9. The method of claim 2, wherein the RTK inhibitor blocks tyrosine autophosphorylation of VEGF receptor 2, VEGF receptor 1, and Tie-2.

10. The method of claim 9, wherein the RTK inhibitor has an IC₅₀ of from 0.1 nM to 200 nM for each of the receptors listed in claim 9.

11. The method of claim 2, wherein the RTK inhibitor blocks tyrosine autophosphorylation of VEGF receptor 2, VEGF receptor 1, and PDGFR.

12. The method of claim 11, wherein the RTK inhibitor has an IC₅₀ of from 0.1 nM to 100 nM for each of the receptors listed in claim 11.

13. The method of claim 2, wherein the RTK inhibitor blocks tyrosine autophosphorylation of VEGF receptor 2 and Tie-2.

14. The method of claim 13, wherein the RTK inhibitor has an IC₅₀ of from 0.1 nM to is 200 nM for each of the receptors listed in claim 13.

15. The method of claim 14, wherein the RTK inhibitor has an IC₅₀ of less than 10 nM for at least one of the receptors listed in claim 13.

16. The method of claim 2, wherein the RTK inhibitor blocks tyrosine autophosphorylation of VEGF receptor 2 and PDGFR.

17. The method of claim 16, wherein the RTK inhibitor has an IC₅₀ of from 0.1 nM to 100 nM for each of the receptors listed in claim 16.

18. The method of claim 17, wherein the RTK inhibitor has an IC₅₀ of less than 10 nM for at least one of the receptors listed in claim 16.

19. The method of claim 2, wherein the RTK inhibitor blocks tyrosine autophosphorylation of VEGF receptor 2, Tie-2, and PDGFR.

20. The method of claim 19, wherein the RTK inhibitor has an IC₅₀ of between 0.1 nM and 200 nM for each of the receptors listed in claim 19.

21. The method of claim 20, wherein the RTK inhibitor has an IC₅₀ of less than 10 nM for at least one of the receptors listed in claim 19.

22. The method of claim 2, wherein said RTK inhibitor is selected from the group consisting of N-[4-(3-amino-1,2-benzisoxazol-4-yl)phenyl]-N′-(3-methylphenyl)urea;
N-[4-(3-amino-1,2-benzisoxazol-4-yl)phenyl]-N′-[2-(trifluoromethyl)phenyl]urea;
N-[4-(3-amino-1,2-benzisoxazol-4-yl)phenyl]-N′-(2-fluoro-5-methylphenyl)urea;
N-[4-(3-amino-1,2-benzisoxazol-4-yl)phenyl]-N′-[3-(trifluoromethyl)phenyl]urea;
N-[4-(3-amino-1,2-benzisoxazol-4-yl)phenyl]-N′-[2-fluoro-5-(trifluoromethyl)phenyl]urea;
N-[4-(3-amino-7-methoxy-1,2-benzisoxazol-4-yl)phenyl]-N′-[2-fluoro-5-(trifluoromethyl)phenyl]urea;
N-[4-(3-amino-7-methoxy-1,2-benzisoxazol-4-yl)phenyl]-N′-(3-methylphenyl)urea;
N-[4-(3-amino-7-methoxy-1,2-benzisoxazol-4-yl)phenyl]-N′-[3-(trifluoromethyl)phenyl]urea;
N-[4-(3-amino-7-methoxy-1,2-benzisoxazol-4-yl)phenyl]-N′-(3-chlorophenyl)urea;
N-[4-(3-amino-7-methoxy-1,2-benzisoxazol-4-yl)phenyl]-N′-(2-fluoro-5-methylphenyl)urea;
N-{4-[3-amino-7-(4-morpholinylmethyl)-1,2-benzisoxazol-4-yl]phenyl}-N′-[2-fluoro-5-(trifluoromethyl)phenyl]urea;
N-{4-[3-amino-7-(4-morpholinylmethyl)-1,2-benzisoxazol-4-yl]phenyl}-N′-[3-(trifluoromethyl)phenyl]urea;
N-{4-[3-amino-7-(4-morpholinylmethyl)-1,2-benzisoxazol-4-yl]phenyl}-N′-(3-chlorophenyl)urea;
N-{4-[3-amino-7-(4-morpholinylmethyl)-1,2-benzisoxazol-4-yl]phenyl}-N′-(3-methylphenyl)urea;
N-{4-[3-amino-7-(4-morpholinylmethyl)-1,2-benzisoxazol-4-yl]phenyl}-N′-(2-fluoro-5-methylphenyl)urea;
N-{4-[3-amino-7-(4-morpholinylmethyl)-1,2-benzisoxazol-4-yl]phenyl}-N′-(3,5-dimethylphenyl)urea;
N-{4-[3-amino-7-(4-morpholinylmethyl)-1,2-benzisoxazol-4-yl]phenyl}-N′-(3-phenoxyphenyl)urea;
N-{4-[3-amino-7-(4-morpholinylmethyl)-1,2-benzisoxazol-4-yl]phenyl}-N′-(3-bromophenyl)urea;
N-(4-{3-amino-7-[2-(4-morpholinyl)ethoxy]-1,2-benzisoxazol-4-yl}phenyl)-N′-[3-(trifluoromethyl)phenyl]urea;
N-(4-{3-amino-7-[2-(4-morpholinyl)ethoxy]-1,2-benzisoxazol-4-yl}phenyl)-N′-(2-fluoro-5-methylphenyl)urea;
N-(4-{3-amino-7-[2-(4-morpholinyl)ethoxy]-1,2-benzisoxazol-4-yl}phenyl)-N′-[2-fluoro-5-(trifluoromethyl)phenyl]urea;
N-(4-{3-amino-7-[2-(4-morpholinyl)ethoxy]-1,2-benzisoxazol-4-yl}phenyl)-N′-(3-methylphenyl)urea;
N-[4-(3-amino-1,2-benzisoxazol-4-yl)phenyl]-N′-(3,5-dimethylphenyl)urea;
N-[4-(3-amino-1,2-benzisoxazol-4-yl)phenyl]-N′-phenylurea;
N-[4-(3-amino-1,2-benzisoxazol-4-yl)phenyl]-N′-(4-methylphenyl)urea;
N-[4-(3-amino-1,2-benzisoxazol-4-yl)phenyl]-N′-(3-cyanophenyl)urea;
N-[4-(3-amino-1,2-benzisoxazol-4-yl)phenyl]-N′-[4-fluoro-3-(trifluoromethyl)phenyl]urea;
N-[4-(3-amino-1,2-benzisoxazol-4-yl)phenyl]-N′-(3-bromophenyl)urea;
N-[4-(3-amino-1,2-benzisoxazol-4-yl)phenyl]-N′-(3-chlorophenyl)urea;
N-[4-(3-amino-1,2-benzisoxazol-4-yl)phenyl]-N′-(3-ethylphenyl)urea;
N-[4-(3-amino-1,2-benzisoxazol-4-yl)phenyl]-N′-[4-(trifluoromethyl)phenyl]urea;
N-[4-(3-amino-1,2-benzisoxazol-4-yl)phenyl]-N′-(3-fluoro-4-methylphenyl)urea;
N-[4-(3-amino-1,2-benzisoxazol-4-yl)phenyl]-N′-(3-fluorophenyl)urea;
N-[4-(3-amino-1,2-benzisoxazol-4-yl)phenyl]-N′-(3,5-difluorophenyl)urea;
N-[4-(3-amino-1,2-benzisoxazol-4-yl)phenyl]-N′-(3-methoxyphenyl)urea;
N-[4-(3-amino-1,2-benzisoxazol-4-yl)phenyl]-N′-(4-methoxyphenyl)urea;
N-[4-(3-amino-1,2-benzisoxazol-4-yl)phenyl]urea;
N-[4-(3-amino-1,2-benzisoxazol-4-yl)phenyl]-N′-(3-nitrophenyl)urea;
N-[4-(3-amino-1,2-benzisoxazol-4-yl)phenyl]-N′-(4-fluorophenyl)urea;
N-[4-(3-amino-1,2-benzisoxazol-4-yl)phenyl]-N′-(2-fluorophenyl)urea;
N-[4-(3-amino-1,2-benzisoxazol-4-yl)phenyl]-N′-(3-chloro-4-fluorophenyl)urea;
N-[4-(3-amino-1,2-benzisoxazol-4-yl)phenyl]-N′-(3-chloro-4-methoxyphenyl)urea;
N-[4-(3-amino-1,2-benzisoxazol-4-yl)phenyl]-N′-[4-(dimethylamino)phenyl]urea;
N-[4-(3-amino-1,2-benzisoxazol-4-yl)phenyl]-N′-[4-(trifluoromethoxy)phenyl]urea;
N-[4-(3-amino-1,2-benzisoxazol-4-yl)phenyl]-N′-[2-(trifluoromethoxy)phenyl]urea;
N-[4-(3-amino-1,2-benzisoxazol-4-yl)phenyl]-N′-[3,5-bis(trifluoromethyl)phenyl]urea;
N-[4-(3-amino-1,2-benzisoxazol-4-yl)phenyl]-N′-(3-chloro-4-methylphenyl)urea;
N-[4-(3-amino-7-methoxy-1,2-benzisoxazol-4-yl)phenyl]-N′-[3,5-bis(trifluoromethyl)phenyl]urea;
N-[4-(3-amino-7-methoxy-1,2-benzisoxazol-4-yl)phenyl]-N′-[4-(trifluoromethoxy)phenyl]urea;
N-[4-(3-amino-7-methoxy-1,2-benzisoxazol-4-yl)phenyl]-N′-(3-fluorophenyl)urea;
N-[4-(3-amino-7-methoxy-1,2-benzisoxazol-4-yl)phenyl]-N′-(3-methoxyphenyl)urea;
N-[4-(3-amino-7-methoxy-1,2-benzisoxazol-4-yl)phenyl]-N′-(3,5-difluorophenyl)urea;
N-[4-(3-amino-7-methoxy-1,2-benzisoxazol-4-yl)phenyl]-N′-(4-methylphenyl)urea;
N-[4-(3-amino-7-methoxy-1,2-benzisoxazol-4-yl)phenyl]-N′-(3-bromophenyl)urea;
N-[4-(3-amino-7-methoxy-1,2-benzisoxazol-4-yl)phenyl]-N′-(3,5-dimethylphenyl)urea;
N-[4-(3-amino-7-methoxy-1,2-benzisoxazol-4-yl)phenyl]-N′-[4-(dimethylamino)phenyl]urea;
N-[4-(3-amino-7-methyl-1,2-benzisoxazol-4-yl)phenyl]-N′-(3-methylphenyl)urea;
N-[4-(3-amino-7-methyl-1,2-benzisoxazol-4-yl)phenyl]-N′-(3-chlorophenyl)urea;
N-[4-(3-amino-7-methyl-1,2-benzisoxazol-4-yl)phenyl]-N′-(2-fluoro-5-methylphenyl)urea;
N-[4-(3-amino-7-methyl-1,2-benzisoxazol-4-yl)phenyl]-N′-[2-fluoro-5-(trifluoromethyl)pheyl]urea;
N-[4-(3-amino-7-methyl-1,2-benzisoxazol-4-yl)phenyl]-N′-[3-(trifluoromethyl)phenyl]urea;
N-[4-(3-amino-7-methyl-1,2-benzisoxazol-4-yl)phenyl]-N′-(3,5-dimethylphenyl)urea;
N-[4-(3-amino-7-methyl-1,2-benzisoxazol-4-yl)phenyl]-N′-(3-ethylphenyl)urea;
N-[4-(3-amino-7-methyl-1,2-benzisoxazol-4-yl)phenyl]-N′-(4-methylphenyl)urea;
N-[4-(3-amino-7-methyl-1,2-benzisoxazol-4-yl)phenyl]-N′-[4-(trifluoromethoxy)phenyl]urea;
N-[4-(3-amino-7-methyl-1,2-benzisoxazol-4-yl)phenyl]-N′-(3-fluoro-4-methylphenyl)urea;
N-[4-(3-amino-7-methyl-1,2-benzisoxazol-4-yl)phenyl]-N′-(3-methoxyphenyl)urea;
N-[4-(3-amino-7-methyl-1,2-benzisoxazol-4-yl)phenyl]-N′-phenylurea;
N-[4-(3-amino-7-methyl-1,2-benzisoxazol-4-yl)phenyl]-N′-[3,5-bis(trifluoromethyl)phenyl]urea;
N-[4-(3-amino-7-methyl-1,2-benzisoxazol-4-yl)phenyl]-N′-(3-bromophenyl)urea;
N-[4-(3-amino-7-methyl-1,2-benzisoxazol-4-yl)phenyl]-N′-(3-fluorophenyl)urea;
N-[4-(3-amino-7-methoxy-1,2-benzisoxazol-4-yl)phenyl]-N′-[4-fluoro-3-(trifluoromethyl)phenyl]urea;
N-[4-(3-amino-7-methoxy-1,2-benzisoxazol-4-yl)phenyl]-N′-(4-fluoro-3-methylphenyl)urea;
N-[4-(3-amino-7-fluoro-1,2-benzisoxazol-4-yl)phenyl]-N′-[3-(trifluoromethyl)phenyl]urea;
N-[4-(3-amino-7-fluoro-1,2-benzisoxazol-4-yl)phenyl]-N′-(3-chlorophenyl)urea;
N-[4-(3-amino-7-fluoro-1,2-benzisoxazol-4-yl)phenyl]-N′-[4-fluoro-3-(trifluoromethyl)phenyl]urea;
N-[4-(3-amino-7-fluoro-1,2-benzisoxazol-4-yl)phenyl]-N′-(3-methylphenyl)urea;
N-[4-(3-amino-7-fluoro-1,2-benzisoxazol-4-yl)phenyl]-N′-[2-fluoro-5-(trifluoromethyl)phenyl]urea;
N-[4-(3-amino-7-fluoro-1,2-benzisoxazol-4-yl)phenyl]-N′-(2-fluoro-5-methylphenyl)urea;
N-{4-[3-amino-7-(trifluoromethoxy)-1,2-benzisoxazol-4-yl]phenyl}-N′-[2-fluoro-5-(trifluoromethyl)phenyl]urea;
N-{4-[3-amino-7-(trifluoromethoxy)-1,2-benzisoxazol-4-yl]phenyl}-N′-[3-(trifluoromethyl)phenyl]urea;
N-{4-[3-amino-7-(trifluoromethoxy)-1,2-benzisoxazol-4-yl]phenyl}-N′-(2-fluoro-5-methylphenyl)urea;
N-{4-[3-amino-7-(trifluoromethoxy)-1,2-benzisoxazol-4-yl]phenyl}-N′-(3-chlorophenyl)urea;
N-{4-[3-amino-7-(trifluoromethoxy)-1,2-benzisoxazol-4-yl]phenyl}-N′-(3-bromophenyl)urea;
N-{4-[3-amino-7-(trifluoromethoxy)-1,2-benzisoxazol-4-yl]phenyl}-N′-[4-fluoro-3-(trifluoromethyl)phenyl]urea;
N-[4-[3-amino-1H-indazol-4-yl]phenyl]-N′-(2-fluoro-5-methoylphenyl)urea;
N-{4-[3-amino-7-(trifluoromethoxy)-1,2-benzisoxazol-4-yl]phenyl}-N′-(4-fluoro-3-methylphenyl)urea;
N-[4-(3-amino-1H-indazol-4-yl)phenyl]-N′-(3-methylphenyl)urea;
N-[4-(3-amino-1H-indazol-4-yl)phenyl]-N′-(3,5-dimethoxyphenyl)urea;
N-[4-(3-amino-1H-indazol-4-yl)phenyl]-N′-(3-chlorophenyl)urea;
N-[4-(3-amino-1H-indazol-4-yl)phenyl]-N′-[3-(trifluoromethyl)phenyl]urea;
N-[4-(3-amino-1H-indazol-4-yl)phenyl]-N′-[2-fluoro-5-(trifluormethyl)phenyl]urea;
N-[4-(3-amino-1H-indazol-4-yl)phenyl]-N′-(3-bromophenyl)urea;
N-[4-(3-amino-1H-indazol-4-yl)phenyl]-N′-(3-bromo-4-methylphenyl)urea;
N-[4-(3-amino-1H-indazol-4-yl)phenyl]-N′-(3-ethylphenyl)urea;
N-[4-(3-amino-1H-indazol-4-yl)phenyl]-N′-phenyl urea;
N-[4-(3-amino-1H-indazol-4-yl)phenyl]-N′-(3-fluoro-4-methylphenyl)urea;
N-[4-(3-amino-1H-indazol-4-yl)phenyl]-N′-(2-fluorophenyl)urea;
N-[4-(3-amino-1H-indazol-4-yl)phenyl]-N′-(4-fluorophenyl)urea;
N-[4-(3-amino-1H-indazol-4-yl)phenyl]-N′-(3-fluorophenyl)urea;
N-[4-(3-amino-1H-indazol-4-yl)phenyl]-N′-(3-hydroxyphenyl)urea;
N-[4-(3-amino-1H-indazol-4-yl)phenyl]-N′-(3-methylphenyl)urea;
N-[4-(3-amino-1H-indazol-4-yl)-2-fluorophenyl]-N′-(2-fluoro-5-methylphenyl)urea;
N-[4-(3-amino-1H-indazol-4-yl)phenyl]-N′-[4-fluoro-3-(trifluoromethyl)phenyl]urea;
N-[4-(3-amino-1H-indazol-4-yl)phenyl]-N′-[2-fluoro-3-(trifluoromethyl)phenyl]urea;
N-[4-(3-amino-1H-indazol-4-yl)phenyl]-N′-(4-bromo-2-fluorophenyl)urea;
N-[4-(3-amino-1H-indazol-4-yl)phenyl]-N′-(5-fluoro-2-methylphenyl)urea;
N-[4-(3-amino-1H-indazol-4-yl)phenyl]-N′-(4-fluoro-3-methylphenyl)urea;
N-[4-(3-amino-1H-indazol-4-yl)phenyl]-N′-[2-fluoro-5-(hydroxymethyl)phenyl]urea;
3-[({[4-(3-amino-1H-indazol-4-yl)phenyl]amino}carbonyl)amino]-4-fluorobenzoic acid; and
Methyl 3-[({[4-(3-amino-1H-indazol-4-yl)phenyl]amino}carbonyl)amino]-4-fluorobenzoate.

23. The method of claim 22, wherein the RTK inhibitor is N-[4-[3-amino-1H-indazol-4-yl)phenyl]-N′-(2-fluoro-5-methylphenyl)urea;

24. The method of claim 1, wherein the composition is administered as an irrigating solution.

25. The method of claim 1, wherein the active agent is an anti-VEGF compound.

26. The method of claim 25, wherein the anti-VEGF compound is selected from the group consisting of ranibizumab, bevacizumab, pegaptanib, VEGF Trap, anecortave acetate, anecortave desacetate, an siRNA molecule targeting VEGF, and rapamycin.
